(19) United States
(12) Patent Application Publication
(10) Pub. No.: US 2007/0265258 A1
(43) Pub. Date: Nov. 15, 2007

(54) Quinazoline derivatives as phosphodiesterase 10 inhibitors

(76) Inventors: Ruiping Liu, Huntington, NY (US); Mark P. Arrington, Westwood, NJ (US); Allen T. Hopper, Mahwah, NJ (US); Richard D. Conticello, Cortlandt Manor, NY (US); Truc M. Nguyen, New York, NY (US); Carla Gauss, White Plains, NY (US); Stephen A. Hitchcock, Westlake Village, CA (US)

Correspondence Address: JONES DAY, 222 EAST 41ST ST, NEW YORK, NY, 10017, US

(21) Appl. No.: 11/714,597

(22) Filed: Mar. 5, 2007

Related U.S. Application Data
(60) Provisional application No. 60/779,856, filed on Mar. 6, 2006.

(51) Int. Cl. A61K 31/5375; A61K 31/517; A61P 25/18; C07D 235/00; C07D 295/00

(52) U.S. Cl. 514/233.5; 514/266.2; 544/116; 544/283

(57) ABSTRACT

The present invention if directed to certain quinazoline compounds that are PDE10 inhibitors, pharmaceutical compounds containing the same and processes for preparing the same. The invention is also directed to methods of treating diseases treatable by PDE10 enzyme such as obesity, non-insulin dependent diabetes, schizophrenia or bipolar disorder, obsessive-compulsive disorder, and the like.

### CROSS-REFERENCE

This application claims the benefit of U.S. Provisional Application No. 60/779,856 filed Mar. 6, 2006, the disclosure of which is incorporated herein by reference in its entirety.

### FIELD OF THE INVENTION

The present invention is directed to certain quinazoline compounds that are PDE10 inhibitors, pharmaceutical compositions containing such compounds and processes for preparing such compounds. The invention is also directed to methods of treating diseases treatable by inhibition of PDE10 enzyme, such as obesity, non-insulin dependent diabetes, schizophrenia, bipolar disorder, obsessive-compulsive disorder, and the like.

### BACKGROUND

Neurotransmitters and hormones, as well as other types of extracellular signals such as light and odors, create intracellular signals by altering the amounts of cyclic nucleotide monophosphates (cAMP and cGMP) within cells. These intracellular messengers alter the functions of many intracellular proteins. Cyclic AMP regulates the activity of cAMP-dependent protein kinase (PKA). PKA phosphorylates and regulates the function of many types of proteins, including ion channels, enzymes, and transcription factors. Downstream mediators of cGMP signaling also include kinases and ion channels. In addition to actions mediated by kinases, cAMP and cGMP bind directly to some cell proteins and directly regulate their activity.

Cyclic nucleotides are produced from the actions of adenylyl cyclase and guanylyl cyclase which convert ATP to cAMP and GTP to cGMP. Extracellular signals, often through the actions of G protein-coupled receptors, regulate the activity of the cyclases. Alternatively, the amount of cAMP and cGMP may be altered by regulating the activity of the enzymes that degrade cyclic nucleotides. Cell homeostasis is maintained by the rapid degradation of cyclic nucleotides after stimulus-induced increases. The enzymes that degrade cyclic nucleotides are called 3′,5′-cyclic nucleotide-specific phosphodiesterases (PDEs).

Eleven PDE gene families (PDE1-PDE11) have been identified based on their distinct amino acid sequences, catalytic and regulatory characteristics, and sensitivity to small molecule inhibitors. These families are coded for by 21 genes; and further multiple splice variants are transcribed from many of these genes. Expression patterns of each of the gene families are distinct. PDEs differ with respect to their affinity for cAMP and cGMP. Activities of different PDEs are regulated by different signals. For example, PDE 1 is stimulated by Ca²⁺/calmodulin. PDE 2 activity is stimulated by cGMP. PDE 3 is inhibited by cGMP. PDE 4 is cAMP specific and is specifically inhibited by rolipram. PDE 5 is cGMP-specific. PDE6 is expressed in retina.

PDE10 sequences were first identified by using bioinformatics and sequence information from other PDE gene families (Fujishige et al., J. Biol. Chem. 274:18438-18445, 1999; Loughney, K. et al., Gene 234:109-117, 1999; Soderling, S. et al., Proc. Natl. Acad. Sci. USA 96:7071-7076, 1999). The PDE10 gene family is distinguished based on its amino acid sequence, functional properties and tissue distribution. The human PDE10 gene is large, over 200 kb, with up to 24 exons coding for each of the splice variants. The amino acid sequence is characterized by two GAF domains (which bind cGMP), a catalytic region, and alternatively spliced N and C termini. Numerous splice variants are possible because of at least three alternative exons encoding N termini and two exons encoding C termini. PDE10A1 is a 779 amino acid protein that hydrolyzes both cAMP and cGMP. The Kₘ values for cAMP and cGMP are 0.05 and 3.0 micromolar, respectively. In addition to human variants, several variants with high homology have been isolated from both rat and mouse tissues and sequence banks.

PDE10 RNA transcripts were initially detected in human testis and brain. Subsequent immunohistochemical analysis revealed that the highest levels of PDE10 are expressed in the basal ganglia. Specifically, striatal neurons in the olfactory tubercle, caudate nucleus and nucleus accumbens are especially enriched in PDE10. Western blots did not reveal the expression of PDE10 in other brain tissues, although immunprecipitation of the PDE10 complex was possible in hippocampal and cortical tissues. This suggests that the expression level of PDE10 in these other tissues is 100-fold less than in striatal neurons. Expression in hippocampus is limited to the cell bodies, whereas PDE10 is expressed in terminals, dendrites and axons of striatal neurons.

The tissue distribution of PDE10 indicates that PDE10 inhibitors can be used to raise levels of cAMP and/or cGMP within cells that express the PDE10 enzyme, especially neurons that comprise the basal ganglia and therefore would be useful in treating a variety of neuropsychiatric conditions involving the basal ganglia such as obesity, non-insulin dependent diabetes, schizophrenia, bipolar disorder, obsessive compulsive disorder, and the like.

### SUMMARY OF THE INVENTION

In one aspect, this invention is directed to a compound of Formula (I): 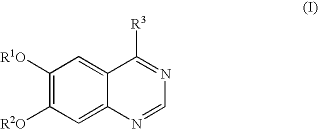
or an individual stereoisomer, a mixture of stereoisomers, or a pharmaceutically acceptable salt thereof, wherein:

R¹ and R² are each independently selected from hydrogen, alkyl, or haloalkyl; and

R³ is:
(i) phenyl, six-membered heteroaryl, or a monocyclic six- or seven-membered heterocyclyl ring substituted with:
   R⁴, where R⁴ is hydrogen, alkyl, halo, haloalkyl, haloalkoxy, cycloalkyl, cycloalkylalkyl, aryl, heteroaryl, heterocyclyl, aralkyl, heteroaralkyl, heterocyclylalkyl, or —XR⁷ (where X is —O—, —CO—, —C(O)O—, —OC(O)—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and
   R⁵ and R⁶, where R⁵ and R⁶ are each independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl provided that at least one of R⁴, R⁵ and R⁶ is not hydrogen;
   and wherein the aromatic or alicyclic ring in R⁴, R⁵, R⁶, and R⁷ is optionally substituted with one to three substituents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substituents independently selected from R^{d} and R^{e} where R^{d} and R^{e} are hydrogen or fluoro;
(ii) pyrrolyl, pyrrolidinyl, 2,4-dioxoimidazolidinyl, or 2-oxopyrrolidinyl substituted with:
   R¹³, where R¹³ is hydrogen, alkyl, halo, haloalkyl, haloalkoxy, cycloalkyl, cycloalkylalkyl, aryl, heteroaryl, heterocyclyl, aralkyl, heteroaralkyl, heterocyclylalkyl, or —XR¹⁶ (where X is —O—, —CO—, —OC(O)—, —C(O)O—, —NR¹⁷CO—, —CONR¹⁸—, —NR¹⁹—, —S—, —SO—, —SO₂—, —NR²⁰SO₂—, or —SO₂NR²¹— where R¹⁷-R²¹ are independently hydrogen, alkyl, hydroxyalkyl, alkoxyalkyl, aryl, aralkyl, heteroaryl, heteroaralkyl, acyl, or heterocyclylalkyl and R¹⁶ is cycloalkyl, cycloalkylalkyl, aryl, heteroaryl, heterocyclyl, aralkyl, heteroaralkyl, or heterocyclylalkyl); and
   R¹⁴ and R¹⁵, where R¹⁴ and R¹⁵ are each independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino, aryl, heteroaryl or heterocyclyl provided that at least one of R¹³, R¹⁴ and R¹⁵ is not hydrogen; and
   wherein the aromatic or alicyclic ring in R¹³, R¹⁴, R¹⁵, and R¹⁶ is optionally substituted with one to three substituents independently selected from R^{f}, R^{g}, and R^{h} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, or disubstituted amino, optionally substituted phenyl, optionally substituted heteroaryl, or optionally substituted heterocyclyl; and additionally substituted with one or two substituents independently selected from Rⁱ and R^{j} where Rⁱ and R^{j} are hydrogen or fluoro;
(iii) a ring of formula (a) 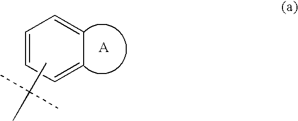
where A is a monocyclic saturated five-, six-, or seven membered heterocyclyl ring and the ring (a) is substituted with:
   R²², where R²² is selected from hydrogen, alkyl, haloalkyl, haloalkoxy, cycloalkyl, cycloalkylalkyl, aryl, heteroaryl, heterocyclyl, aralkyl, heteroaralkyl, heterocyclylalkyl, or —XR²⁵ (where X is —O—, —CO—, —C(O)O—, —OC(O)—, —NR²⁶CO—, —CONR²⁷—, —NR²⁸—, —S—, —SO—, —SO₂—, —NR²⁹SO₂—, or —SO₂NR³⁰— where R²⁶-R³⁰ are independently hydrogen, alkyl, hydroxyalkyl, alkoxyalkyl, aryl, aralkyl, heteroaryl, heteroaralkyl, acyl, or heterocyclylalkyl and R²⁵ is hydrogen, alkyl, haloalkyl, cycloalkyl, cycloalkylalkyl, aryl, heteroaryl, heterocyclyl, aralkyl, heteroaralkyl, or heterocyclylalkyl); and
   R²³ and R²⁴, where R²³ and R²⁴ are each independently selected from hydrogen, alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino, aryl, heteroaryl or heterocyclyl; and
   wherein the aromatic or alicyclic ring in R²², R²³, R²⁴, and R²⁵ is optionally substituted with one to three substituents independently selected from R^{k}, R^{l}, and R^{m} which alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, or disubstituted amino, optionally substituted phenyl, optionally substituted heteroaryl, or optionally substituted heterocyclyl; and additionally substituted with one or two substituents independently selected from Rⁿ and R^{o} where Rⁿ and R^{o} are hydrogen or fluoro provided that at least one of R²², R²³ and R²⁴ is not hydrogen; or
(iv) a ring of formula (b), (c), or (d): 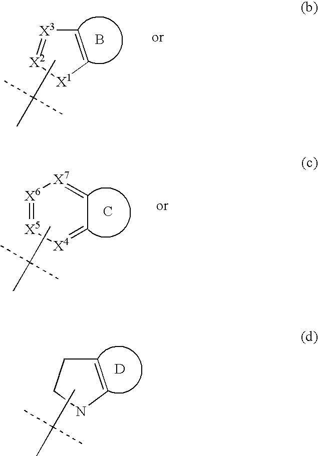
where:
   X¹, X², and X³ are each independently carbon, nitrogen, oxygen or sulfur, provided that at least two of X¹, X², and X³ are other than carbon;
   X⁴, X⁵, X⁶ and X⁷ are each independently carbon or nitrogen, provided that at least two of X⁴, X⁵, X⁶ and X⁷ are other than carbon; and
   B, C, and D are phenyl, a five- or six-membered heteroaryl ring (wherein the five-membered heteroaryl ring contains one or two heteroatoms independently selected from nitrogen, oxygen, and sulfur and the six-membered heteroaryl ring contains one or two nitrogen atoms, the rest of the ring atoms being carbon), or a five-, six-, or seven-membered heterocyclyl ring; and
wherein rings (b) and (c) are substituted with:
   R³¹, where R³¹ is cycloalkyl, cycloalkylalkyl, aryl, heteroaryl, heterocyclyl, aralkyl, heteroaralkyl, heterocyclylalkyl, or —XR³⁴ (where X is —O—, —CO—, —OC(O)—, —C(O)O—, —NR³⁵CO—, —CONR³⁶—, —NR³⁷—, —S—, —SO—, —SO₂—, —NR³⁸SO₂—, or —SO₂NR³⁹— where R³⁵-R³⁹ are independently hydrogen, alkyl, hydroxyalkyl, alkoxyalkyl, aryl, aralkyl, heteroaryl, heteroaralkyl, acyl, or heterocyclylalkyl and R³⁴ is cycloalkyl, cycloalkylalkyl, aryl, heteroaryl, heterocyclyl, aralkyl, heteroaralkyl, or heterocyclylalkyl); and
   R³² and R³³, where R³² and R³³ are each independently selected from hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino, aryl, heteroaryl or heterocyclyl provided that at least one of R³, R³² and R³³ is not hydrogen; and
   wherein the aromatic or alicyclic ring in R³¹, R³², R³³, and R³⁴ is optionally substituted with one to three substituents independently selected from R^{p}, R^{q}, and R^{r} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfonyl, monosubstituted amino, or, disubstituted amino, optionally substituted phenyl, optionally substituted heteroaryl, or optionally substituted heterocyclyl; and additionally substituted with one or two substituents independently selected from R^{s} and R^{t} where R^{s} and R^{t} are hydrogen or fluoro; and

provided that:
(i) R³ is not disubstituted piperidin-1-yl where one substituent is substituted or unsubstituted aryl or heteroaryl, and the other substituent is alkyl, carboxy, alkoxycarbonyl, cyano, hydroxyl, alkoxy, haloalkoxy, pyridin-2-yloxy, —COR, —CONRR′, —COOR, —OR or —NRR′ (where R and R′ are independently hydrogen, alkyl, or unsubstituted aryl), or —NHCOR (where R is alkyl, haloalkyl, or unsubstituted aryl);
(ii) when R³ is pyrrolidin-1-yl, R¹³ is not —XR¹⁶ where X is —O— and R¹⁶ is substituted or unsubstituted aryl or heteroaryl;
(iii) R³ is not (a) monosubstituted phenyl wherein the substituent is hydroxy, nitro, halo, alkoxy, haloalkyl, or unsubstituted aryl; (b) disubstituted phenyl wherein the substituents are independently selected from halo or alkoxy; (c) monosubstituted pyridinyl wherein the substituent is selected from halo or alkoxy; (d) 2,6-dimethylmorpholinyl, or (e) 5-amino-2-phenylcarbonylaminopyrimidinyl;
(iv) R³ is not monosubstituted piperidinyl wherein the substituent is alkyl, hydroxy, carboxy, alkoxycarbonyl, hydroxyalkyl, haloalkyl, substituted or unsubstituted aryl or heteroaryl, substituted or unsubstituted, saturated or unsaturated heterocyclyl or heterocyclylalkyl wherein the heterocyclyl ring contains two ring atoms that are heteroatom selected from N, O, or S(O)ₙ, where n is an integer from 0 to 2, the remaining ring atoms being C, where one or two ring carbon atoms can optionally be replaced by a —CO— group and the heterocyclic ring is optionally fused to a phenyl provided that when the heterocyclyl ring is bicyclic, the bicyclic heterocyclyl ring is attached to the piperidinyl ring via the non-phenyl portion of the ring; —COR (where R is alkyl or unsubstituted aryl), —COOR (where R is unsubstituted aryl), —CONRR′ (where R is hydrogen, alkyl, or unsubstituted aryl, and R′ is unsubstituted aryl), —NRCOR (where R is hydrogen, alkyl, or unsubstituted aryl and R′ is alkyl, haloalkyl, unsubstituted aryl, 4-acetylaminophenyl, piperidin-1-yl, piperidin-1-ylalkyl, or pyridinyl); —NRSO₂R′ (where R is hydrogen or alkyl and R′ is alkyl, 4-acetylaminophenyl, or pyridinyl); —NRR′ (where R is hydrogen, alkyl, or unsubstituted aryl and R′ is alkyl, 2-aminoethyl, 2-benzylaminoethyl, unsubstituted aryl, or pyridinylmethyl); -(alkylene)NRR′ ([where R is hydrogen or alkyl and R′ is hydrogen, alkyl or —COR″ where R″ is alkyl); phenyl (optionally substituted with haloalkyl or alkoxy); substituted or unsubstituted indolinyl, oxazolyl, benzo[d]oxazolyl, oxiranyl, 1H-benzo[d]imidazolyl, 1H-benzo[d][1,2,3]triazolyl, pyridin-2-yloxy, tetrahydronaphthalenyl, or 4H-1,2,4-triazolylalkyl; or piperidin-4-ylalkyl substituted with dialkoxyquinazoline;
(v) R³ is not disubstituted piperidinyl where one of the substituents is alkyl or hydroxy and the other substitutent is hydroxyalkyl, haloalkyl, 1,1-dioxoisothiazolidinylalkyl or 1H-benzo[d]imidazolyl-2(3H)-one, wherein each of these rings is optionally substituted with one or two alkyl, and the 1H-benzo[d]imidazolyl-2(3H)-one is attached to the piperidinyl ring via the non-phenyl portion of the ring;
(vi) R³ is not monosubstituted or disubstituted piperazin-4-yl or homopiperazin-4-yl where the substitutent(s) is alkyl; or R³ is not piperazin-4-yl or homopiperazin-4-yl where R⁵ is hydrogen, R⁶ is hydrogen or alkyl and R⁴ is other than hydrogen and at least one of R⁴ and R⁶ is located at the N−1 nitrogen of the piperazine or homopiperazine ring.

In a second aspect, this invention is directed to a pharmaceutical composition comprising at least a compound of Formula (I) or a pharmaceutically acceptable salt or mixtures thereof and a pharmaceutically acceptable expicient.

In a third aspect, this invention is directed to a method of treating a disorder treatable by inhibition of PDE10 in a patient which method comprises administering to the patient a pharmaceutical composition comprising at least a compound of Formula (I): 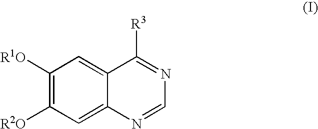
or an individual stereoisomer, a mixture of stereoisomers, or a pharmaceutically acceptable salt thereof; or mixtures thereof, wherein:

R¹ and R² are each independently selected from hydrogen, alkyl, or haloalkyl; and

R³ is:
(i) phenyl, six-membered heteroaryl, or a monocyclic six- or seven-membered heterocyclyl ring substituted with:
   R⁴, where R⁴ is hydrogen, alkyl, halo, haloalkyl, haloalkoxy, cycloalkyl, cycloalkylalkyl, aryl, heteroaryl, heterocyclyl, aralkyl, heteroaralkyl, heterocyclylalkyl, or —XR⁷ (where X is —O—, —CO—, —C(O)O—, —OC(O)—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and
   R⁵ and R⁶, where R⁵ and R⁶ are each independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl provided that at least one of R⁴, R⁵ and R⁶ is not hydrogen;
   and wherein the aromatic or alicyclic ring in R⁴, R⁵, R⁶, and R⁷ is optionally substituted with one to three substituents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substituents independently selected from R^{d} and R^{e} where R^{d} and R^{e} are hydrogen or fluoro;
(ii) pyrrolyl, pyrrolidinyl, 2,4-dioxoimidazolidinyl, or 2-oxopyrrolidinyl substituted with:
   R¹³, where R¹³ is hydrogen, alkyl, halo, haloalkyl, haloalkoxy, cycloalkyl, cycloalkylalkyl, aryl, heteroaryl, heterocyclyl, aralkyl, heteroaralkyl, heterocyclylalkyl, or —XR¹⁶ (where X is —O—, —CO—, —OC(O)—, —C(O)O—, —NR¹⁷CO—, —CONR¹⁸—, —NR¹⁹—, —S—, —SO—, —SO₂—, —NR¹⁰SO₂—, or —SO₂NR²¹— where R¹⁷-R²¹ are independently hydrogen, alkyl, hydroxyalkyl, alkoxyalkyl, aryl, aralkyl, heteroaryl, heteroaralkyl, acyl, or heterocyclylalkyl and R¹⁶ is cycloalkyl, cycloalkylalkyl, aryl, heteroaryl, heterocyclyl, aralkyl, heteroaralkyl, or heterocyclylalkyl); and
   R¹⁴ and R¹⁵, where R¹⁴ and R¹⁵ are each independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino, aryl, heteroaryl or heterocyclyl provided that at least one of R¹³, R¹⁴ and R¹⁵ is not hydrogen; and
   wherein the aromatic or alicyclic ring in R³, R⁴, R¹⁵, and R¹⁶ is optionally substituted with one to three substituents independently selected from R^{f}, R^{g}, and R^{h} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino, optionally substituted phenyl, optionally substituted heteroaryl, or optionally substituted heterocyclyl; and additionally substituted with one or two substituents independently selected from Rⁱ and R^{j} where Rⁱ and R^{j} are hydrogen or fluoro;
(iii) a ring of formula (a) 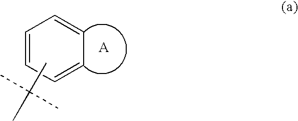
where A is a monocyclic saturated five-, six-, or seven membered heterocyclyl ring and the ring (a) is substituted with:
   R²², where R²² is selected from hydrogen, alkyl, haloalkyl, haloalkoxy, cycloalkyl, cycloalkylalkyl, aryl, heteroaryl, heterocyclyl, aralkyl, heteroaralkyl, heterocyclylalkyl, or —XR²⁵ (where X is —O—, —CO—, —C(O)O—, —OC(O)—, —NR²⁶CO—, —CONR²⁷—, —NR²⁸—, —S—, —SO—, —SO₂—, —NR²⁹SO₂—, or —SO₂NR³⁰— where R²⁶-R³⁰ are independently hydrogen, alkyl, hydroxyalkyl, alkoxyalkyl, aryl, aralkyl, heteroaryl, heteroaralkyl, acyl, or heterocyclylalkyl and R²⁵ is hydrogen, alkyl, haloalkyl, cycloalkyl, cycloalkylalkyl, aryl, heteroaryl, heterocyclyl, aralkyl, heteroaralkyl, or heterocyclylalkyl); and
   R²³ and R²⁴, where R²³ and R²⁴ are each independently selected from hydrogen, alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino, aryl, heteroaryl or heterocyclyl; and
   wherein the aromatic or alicyclic ring in R²², R²³, R²⁴, and R²⁵ is optionally substituted with one to three substituents independently selected from R^{k}, R^{l}, and R^{m} which alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino, optionally substituted phenyl, optionally substituted heteroaryl, or optionally substituted heterocyclyl; and additionally substituted with one or two substituents independently selected from Rⁿ and R^{o} where Rⁿ and R^{o} are hydrogen or fluoro provided that at least one of R²², R²³ and R²⁴ is not hydrogen; or
(iv) a ring of formula (b), (c), or (d): 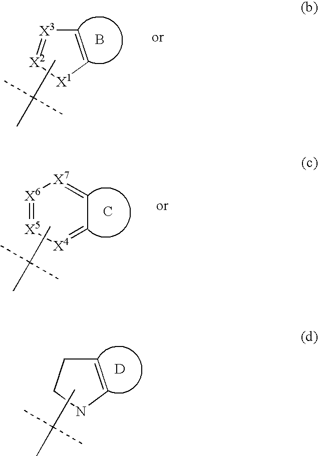
where:
   X¹, X², and X³ are each independently carbon, nitrogen, oxygen or sulfur, provided that at least two of X¹, X², and X³ are other than carbon;
   X⁴, X⁵, X⁶ and X⁷ are each independently carbon or nitrogen provided that at least two of X⁴, X⁵, X⁶ and X⁷ are other than carbon; and
   B, C, and D are phenyl, a five- or six-membered heteroaryl ring (wherein the five-membered heteroaryl ring contains one or two heteroatoms independently selected from nitrogen, oxygen, and sulfur and the six-membered heteroaryl ring contains one or two nitrogen atoms, the rest of the ring atoms being carbon), or a five-, six-, or seven-membered heterocyclyl ring; and
wherein rings (b) and (c) are substituted with:
   R³¹, where R³¹ is cycloalkyl, cycloalkylalkyl, aryl, heteroaryl, heterocyclyl, aralkyl, heteroaralkyl, heterocyclylalkyl, or —XR³⁴ (where X is —O—, —CO—, —OC(O)—, —C(O)O—, —NR³⁵CO—, —CONR³⁶—, —NR³⁷—, —S—, —SO—, —SO₂—, —NR³⁸SO₂—, or —SO₂NR³⁹— where R³⁵-R³⁹ are independently hydrogen, alkyl, hydroxyalkyl, alkoxyalkyl, aryl, aralkyl, heteroaryl, heteroaralkyl, acyl, or heterocyclylalkyl and R³⁴ is cycloalkyl, cycloalkylalkyl, aryl, heteroaryl, heterocyclyl, aralkyl, heteroaralkyl, or heterocyclylalkyl); and
   R³² and R³³, where R³² and R³³ are each independently selected from hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino, aryl, heteroaryl or heterocyclyl provided that at least one of R³¹, R³² and R³³ is not hydrogen; and
   wherein the aromatic or alicyclic ring in R³¹, R³², R³³, and R³⁴ is optionally substituted with one to three substituents independently selected from R^{p}, R^{q}, and R^{r} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfonyl, monosubstituted amino, or, disubstituted amino, optionally substituted phenyl, optionally substituted heteroaryl, or optionally substituted heterocyclyl; and additionally substituted with one or two substituents independently selected from R^{s} and R^{t} where R^{s} and R^{t} are hydrogen or fluoro; and

provided that:
(i) R³ is not disubstituted piperidin-1-yl where one substituent is substituted or unsubstituted aryl or heteroaryl, and the other substituent is alkyl, carboxy, alkoxycarbonyl, cyano, hydroxyl, alkoxy, haloalkoxy, pyridin-2-yloxy, —COR, —CONRR′, —COOR, —OR or —NRR′ (where R and R′ are independently hydrogen, alkyl, or unsubstituted aryl), or —NHCOR (where R is alkyl, haloalkyl, or unsubstituted aryl); or
(ii) when R³ is pyrrolidin-1-yl, R¹³ is not —XR¹⁶ where X is —O— and R¹⁶ is substituted or unsubstituted aryl or heteroaryl.

In one embodiment, the disease is obesity, non-insulin dependent diabetes, Huntington's disease, schizophrenia, bipolar disorder, and obsessive-compulsive disorder.

### DETAILED DESCRIPTION OF THE INVENTION

Definitions

Unless otherwise stated, the following terms used in the specification and claims are defined for the purposes of this Application and have the following meanings.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alicyclic” means a non-aromatic ring, e.g, cycloalkyl or heterocyclyl ring.

“Alkylene” means a linear saturated divalent hydrocarbon radical of one to six carbon atoms or a branched saturated divalent hydrocarbon radical of three to six carbon atoms unless otherwise stated, e.g., methylene, ethylene, propylene, 1-methylpropylene, 2-methylpropylene, butylene, pentylene, and the like.

“Alkylthio” means a —SR radical where R is alkyl as defined above, e.g., methylthio, ethylthio, and the like.

“Alkylsulfonyl” means a —SO₂R radical where R is alkyl as defined above, e.g., methylsulfonyl, ethylsulfonyl, and the like.

“Amino” means —NH₂.

“Alkylamino” means an —NHR radical where R is alkyl as defined above, e.g., methylamino, ethylamino, propylamino, or 2-propylamino, and the like.

“Alkoxy” means an —OR radical where R is alkyl as defined above, e.g., methoxy, ethoxy, propoxy, or 2-propoxy, n-, iso-, or tert-butoxy, and the like.

“Alkoxycarbonyl” means a —C(O)OR radical where R is alkyl as defined above, e.g., methoxycarbonyl, ethoxycarbonyl, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Alkoxyalkyloxy” means an —OR radical where R is alkoxyalkyl as defined above, e.g., methoxyethoxy, 2-ethoxyethoxy, and the like.

“Aminoalkyl” means a linear monovalent hydrocarbon radical of one to six carbon atoms or a branched monovalent hydrocarbon radical of three to six carbons substituted with at least one, preferably one or two, —NRR′ where R is hydrogen, alkyl, or —COR^{a} where R^{a} is alkyl, and R′ is selected from hydrogen, alkyl, hydroxyalkyl, alkoxyalkyl, aryl, aralkyl, heteroaryl, heteroaralkyl, or haloalkyl, each as defined herein, e.g, aminomethyl, methylaminoethyl, 2-ethylamino-2-methylethyl, 1,3-diaminopropyl, dimethylaminomethyl, diethylaminoethyl, acetylaminopropyl, and the like.

“Aminoalkoxy” means an —OR radical where R is aminoalkyl as defined above, e.g., 2-aminoethoxy, 2-dimethylaminopropoxy, and the like.

“Aminocarbonyl” means a —CONRR′ radical where R is independently hydrogen, alkyl, hydroxyalkyl, alkoxyalkyl, or aminoalkyl and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined above, e.g., —CONH₂, methylaminocarbonyl, 2-dimethylaminocarbonyl, and the like.

“Aminosulfinyl” means a —SONRR′ radical where R is independently hydrogen, alkyl, hydroxyalkyl, alkoxyalkyl, or aminoalkyl and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined above, e.g., methylaminosulfinyl, 2-dimethylaminosulfinyl, and the like.

“Aminosulfonyl” means a —SO₂NRR′ radical where R is independently hydrogen, alkyl, hydroxyalkyl, alkoxyalkyl, or aminoalkyl and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined above, e.g., —SO₂NH₂, methylaminosulfonyl, 2-dimethylaminosulfonyl, and the like.

“Acyl” means a —COR radical where R is alkyl, haloalkyl, cycloalkyl, cycloalkylalkyl, aryl, aralkyl, heteroaryl, heteroaralkyl, heterocyclyl, or heterocyclylalkyl, each as defined above, e.g., acetyl, propionyl, benzoyl, pyridinylcarbonyl, and the like.

“Acylamino” means a —NHCOR radical where R is alkyl, haloalkyl, cycloalkyl, cycloalkylalkyl, aryl, aralkyl, heteroaryl, heteroaralkyl, heterocyclyl, or heterocyclylalkyl, each as defined above, e.g., acetylamino, propionylamino, and the like.

“Aryl” means a monovalent monocyclic or bicyclic aromatic hydrocarbon radical of 6 to 12 ring atoms, e.g., phenyl or naphthyl.

“Aralkyl” means an -(alkylene)-R radical where R is aryl as defined above.

“Cycloalkyl” means a cyclic saturated monovalent bridged or non-bridged hydrocarbon radical of three to ten carbon atoms, e.g., cyclopropyl, cyclobutyl, cyclopentyl, cyclohexyl, or adamantyl.

“Cycloalkylalkyl” means an -(alkylene)-R radical where R is cycloalkyl as defined above; e.g., cyclopropylmethyl, cyclobutylmethyl, cyclopentylethyl, or cyclohexylmethyl, and the like.

“Cycloalkyloxy” means a —OR radical where R is cycloalkyl as defined above. Exemplary cycloalkyloxy groups include, e.g., cyclopropyloxy, cyclobutyloxy, cyclopentyloxy, cyclohexyloxy, and the like.

“Cycloalkylalkyloxy” means an —OR radical where R is cycloalkylalkyl as defined above. Exemplary cycloalkylalkyloxy groups include, e.g., cyclopropylmethyloxy, cyclobutylmethyloxy, cyclopentylethyloxy, cyclohexylmethyloxy, and the like.

“Carboxy” means —COOH.

“Disubstituted amino” means a —NRR′ radical where R and R′ are independently alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl, each as defined above, e.g., dimethylamino, phenylmethylamino, and the like. When R and R′ are alkyl, they are also referred to herein as dialkylamino.

“Halo” means fluoro, chloro, bromo, and iodo, preferably fluoro or chloro.

“Haloalkyl” means alkyl substituted with one or more halogen atoms, preferably one to five halogen atoms, preferably fluorine or chlorine, including those substituted with different halogens, e.g., —CH₂Cl, —CF₃, —CHF₂, —CF₂CF₃, —CF(CH₃)₃, and the like.

“Haloalkoxy” means a —OR radical where R is haloalkyl as defined above e.g., —OCF₃, —OCHF₂, and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkoxy” or “hydroxyalkyloxy” means a —OR radical where R is hydroxyalkyl as defined above.

“Heterocyclyl” means a saturated or unsaturated monovalent monocyclic group of 3 to 8 ring atoms in which one or two ring atoms are heteroatom independently selected from N, O, or S(O)ₙ, where n is an integer from 0 to 2, the remaining ring atoms being C. Additionally, one or two ring carbon atoms can optionally be replaced by a —CO— group and the heterocyclic ring may be fused to phenyl or heteroaryl ring, provided that the entire ring is not aromatic. Unless stated otherwise, the fused heterocyclyl ring can be attached at any ring atom. More specifically the term heterocyclyl includes, but is not limited to, pyrrolidino, piperidino, 2-oxopyrrolidinyl, 2-oxopiperidinyl, morpholino, piperazino, tetrahydropyranyl, thiomorpholino, homopiperidino, and the like. When the heterocyclyl ring has five, six or seven ring atoms and is not fused to phenyl or heteroaryl ring, it is referred to herein as “monocyclic five- six-, or seven membered heterocyclyl ring or five- six-, or seven membered heterocyclyl ring”. When the heterocyclyl ring is unsaturated it can contain one or two double bonds provided that the ring is not aromatic.

“Heterocyclylalkyl” means an -(alkylene)-R radical where R is heterocyclyl ring as defined above, e.g., tetraydrofuranylmethyl, piperazinylmethyl, morpholinylethyl, and the like.

“Heteroaryl” means a monovalent monocyclic or bicyclic aromatic radical of 5 to 10 ring atoms where one, two, or three, ring atoms are heteroatom independently selected from N, O, or S, the remaining ring atoms being carbon.

“Heteroaralkyl” means an -(alkylene)-R radical where R is heteroaryl as defined above.

“Monosubstituted amino” means a —NHR radical where R is alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl, each as defined above, e.g., methylamino, 2-phenylamino, hydroxyethylamino, and the like.

The present invention also includes prodrugs of compounds of Formula (I). The term prodrug is intended to represent covalently bonded carriers, which are capable of releasing the active ingredient of Formula (I) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups in vivo or by routine manipulation. Prodrugs of compounds of Formula (I) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) are also within the scope of this invention.

The present invention also includes protected derivatives of compounds of Formula (I). For example, when compounds of Formula (I) contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1999, the disclosure of which is incorporated herein by reference in its entirety. The protected derivatives of compounds of Formula (I) can be prepared by methods well known in the art.

A “pharmaceutically acceptable salt” of a compound means a salt that is pharmaceutically acceptable and that possesses the desired pharmacological activity of the parent compound. Such salts include, for example, acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

A “pharmaceutically acceptable salt” can include, for example, salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. All chiral, diastereomeric, racemic forms are within the scope of this invention, unless the specific stereochemistry or isomeric form is specifically indicated.

Certain compounds of Formula (I) can exist as tautomers and/or geometric isomers. All possible tautomers and cis and trans isomers, individual forms and mixtures thereof, are within the scope of this invention. Additionally, as used herein the term alkyl includes all the possible isomeric forms of said alkyl group albeit only a few examples are set forth. Furthermore, when the cyclic groups such as aryl, heteroaryl, heterocyclyl are substituted, they include all the positional isomers albeit only a few examples are set forth. Furthermore, all polymorphic forms and hydrates of a compound of Formula (I) are within the scope of this invention.

“Oxo” or “carbonyl” means ═(O) group.

“Optional” or “optionally” means that the subsequently described event or circumstance may but need not occur, and that the description includes instances where the event or circumstance occurs and instances in which it does not. For example, “heterocyclyl group optionally mono- or di-substituted with an alkyl group” means that the alkyl may but need not be present, and the description includes situations where the heterocyclyl group is mono- or disubstituted with alkyl and situations where the heterocyclyl group is not substituted with alkyl.

“Optionally substituted phenyl” means a phenyl ring optionally substituted with one, two, or three substituents independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, carboxy, cycloalkyl, cycloalkylalkyl, cycloalkoxy, cycloalkylalkyloxy, sulfinyl, or sulfonyl, each as defined herein.

“Optionally substituted heteroaryl” means a monovalent monocyclic or bicyclic aromatic radical of 5 to 10 ring atoms where one or more, preferably one, two, or three, ring atoms are heteroatoms independently selected from N, O, or S, the remaining ring atoms being carbon that is optionally substituted with one, two, or three substituents independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, or carboxy, cycloalkyl, cycloalkylalkyl, cycloalkoxy, cycloalkylalkyloxy, sulfinyl, or sulfonyl, each as defined herein. More specifically the term optionally substituted heteroaryl includes, but is not limited to, optionally substituted pyridyl, pyrrolyl, imidazolyl, thienyl, furanyl, indolyl, quinolyl, pyrazinyl, pyrimidinyl, pyridazinyl, oxazolyl, isoxazolyl, benzoxazolyl, quinolinyl, isoquinolinyl, benzopyranyl, and thiazolyl which are substituted or unsubstituted as stated above.

“Optionally substituted heterocyclyl” means a saturated or unsaturated monovalent cyclic group of 3 to 8 ring atoms in which one or two ring atoms are heteroatoms selected from N, O, or S(O)ₙ, where n is an integer from 0 to 2, the remaining ring atoms being C. One or two ring carbon atoms can optionally be replaced by a —CO— group and is optionally substituted with one, two, or three substituents independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, or carboxy, cycloalkyl, cycloalkylalkyl, cycloalkoxy, cycloalkylalkyloxy, sulfinyl, or sulfonyl, each as defined herein.

More specifically the term “optionally substituted heterocyclyl” includes, but is not limited to, optionally substituted pyrrolidino, piperidino, morpholino, piperazino, tetrahydropyranyl, and thiomorpholino which are substituted or unsubstituted as stated above.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Sulfinyl” means a —SOR radical where R is alkyl, haloalkyl, aryl, aralkyl, heteroaryl, heteroaralkyl, heterocyclyl, or heterocyclylalkyl, each as defined above, e.g., methylsulfinyl, phenylsulfinyl, benzylsulfinyl, pyridinylsulfinyl, and the like.

“Sulfonyl” means a —SO₂R radical where R is alkyl, haloalkyl, aryl, aralkyl, heteroaryl, heteroaralkyl, heterocyclyl, or heterocyclylalkyl, each as defined above, e.g., methylsulfonyl, phenylsulfonyl, benzylsulfonyl, pyridinylsulfonyl, and the like.

The expression “wherein the aromatic or alicyclic ring in . . . is optionally substituted with one to three substitutents independently selected from . . . ” in this Application means that all rings that are part of the listed groups are optionally independently substituted. For example, with respect to R⁴, the expression “wherein the aromatic or alicyclic ring in R⁴, R⁵, R⁶, and R⁷ is optionally substituted with one to three substitutents independently selected from R^{a}, R^{b}, and R^{c}” means that when R⁴ is cycloalkyl, cycloalkylalkyl, aryl, heteroaryl, heterocyclyl, aralkyl, heteroaralkyl, heterocyclylalkyl, or —XR⁷ (where X is —O—, —CO—, —C(O)O—, —OC(O)—, —NR⁸CO—, —CONR⁹—, —NR¹⁰, —S—, —SO—, —SO₂—, —NR¹¹SO₂—, or —SO₂NR¹²— where R⁸-R¹² are independently aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl), the rings recited in the definitions of R⁴, R⁷, R⁸, R⁹, R¹⁰, R¹¹ and R¹² are each optionally substituted.

“Treating” or “treatment” of a disease includes:

(1) preventing the disease, i.e. causing the clinical symptoms of the disease not to develop in a mammal that may be exposed to or predisposed to the disease but does not yet experience or display symptoms of the disease;

(2) inhibiting the disease, i.e., arresting or reducing the development of the disease or its clinical symptoms; or

(3) relieving the disease, i.e., causing regression of the disease or its clinical symptoms.

A “therapeutically effective amount” means the amount of a compound of Formula (I) that, when administered to a mammal for treating a disease, is sufficient to effect such treatment for the disease. The “therapeutically effective amount” will vary depending on the compound, the disease and its severity and the age, weight, etc., of the mammal to be treated.

The rings recited in the provisos in the Summary of the Invention have the following structures:

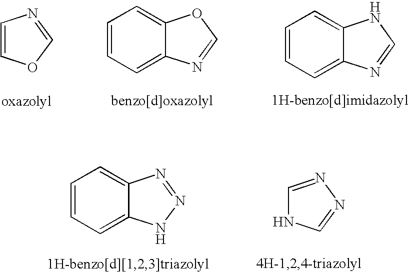

### EMBODIMENTS

In one aspect, provided herein is a compound of Formula (I) or an individual stereoisomer, a mixture of stereoisomers, or a pharmaceutically acceptable salt thereof, as are described in the Summary of the Invention.

(1) In one embodiment, this invention is directed to a compound of Formula (I) where R¹ and R² are alkyl. Within this embodiment, one group of compounds is that wherein R¹ and R² are methyl. Within this embodiment, another group of compounds is that wherein R¹ is ethyl, propyl or butyl and R² is methyl.

(2) In another embodiment, this invention is directed to a compound of Formula (I) where R¹ and R² are haloalkyl, preferably trifluoromethyl or difluoromethyl.

(3) In another embodiment, this invention is directed to a compound of Formula (I) where one of R¹ and R² is alkyl and the other is haloalkyl, preferably one if methyl or ethyl and the other is trifluoromethyl or difluoromethyl.

(a) Within the above embodiments (1), (2) and (3), including the subgroups contained therein, one group of compounds of Formula (I) is that wherein R³ is a monocyclic six- or seven-membered heterocyclyl ring substituted with R⁴, R⁵ and R⁶ as defined below.

R⁴ is selected from aryl, heteroaryl, heterocyclyl, aralkyl, heteroaralkyl, heterocyclylalkyl, cycloalkyl, or —XR⁷ (where X is —O—, —CO—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl).

R⁵ is alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl.

R⁶ is hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino, preferably hydrogen.

The aromatic or alicyclic ring in R⁴, R⁵, R⁶, and R⁷ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e} where R^{d} and R^{e} are hydrogen or fluoro provided that: (i) when R¹ and R² are hydrogen or alkyl, R³ is piperidin-1-yl, azepan-1-yl, 2,3,4,7-tetrahydro-1H-azepin-1-yl, or 1,2,3,6-tetrahydropyridin-1-yl, one of R⁵ and R⁶ is hydrogen or alkyl and the other of R⁵ and R⁶ is hydrogen, then R⁴ is not —NR⁸COR⁷ or —NR¹¹SO₂R⁷ where R⁸ and R¹¹ are hydrogen, alkyl, hydroxyalkyl, alkoxyalkyl, aryl, heteroaryl, aralkyl, heteroaryl, heteroaralkyl, or saturated monocyclic heterocyclylalkyl containing one nitrogen atom wherein the aryl or heteroaryl ring including the ring in aralkyl and heteroaralkyl are optionally substituted with alkyl, alkoxy, hydroxy, halo or acetamido and R⁷ is aryl, heteroaryl, aralkyl, heteroaralkyl or saturated monocyclic heterocyclyl or heterocyclylalkyl containing one nitrogen atom and wherein the aryl or heteroaryl ring including the one in aralkyl and heteroaralkyl are optionally substituted with alkyl, alkoxy, hydroxy, halo or acetamido; (ii) when R¹ and R² are hydrogen or alkyl, R³ is piperidin-1-yl, azepan-1-yl, 2,3,4,7-tetrahydro-1H-azepin-1-yl, or 1,2,3,6-tetrahydropyridin-1-yl, R⁴ is hydrogen and one of R⁵ and R⁶ is hydrogen or alkyl, then the other of R⁵ and R⁶ is not —NRR′ [where R is hydrogen, alkyl, alkyl substituted with amino, monoalkyl or dialkylamino, hydroxyalkyl, alkoxyalkyl, aryl, heteroaryl, aralkyl, heteroaralkyl, or saturated monocyclic heterocyclylalkyl containing one nitrogen atom wherein the aryl or heteroaryl ring including the ring in aralkyl and heteroaralkyl are optionally substituted with alkyl, alkoxy, hydroxy, halo or acetamido and R′ is —COR or —SO₂R (where R is alkyl, aryl, hetereoaryl, aralkyl, heteroaralkyl or saturated monocyclic heterocyclyl or heterocyclylalkyl containing one nitrogen atom and wherein the aryl or heteroaryl ring including the one in aralkyl and heteroaralkyl are optionally substituted with alkyl, alkoxy, hydroxy, halo or acetamido)]; and (iii) when R¹ and R² are hydrogen or alkyl, R³ is piperidin-1-yl, azepan-1-yl, 2,3,4,7-tetrahydro-1H-azepin-1-yl, or 1,2,3,6-tetrahydropyridin-1-yl, one of R⁵ and R⁶ is hydrogen or alkyl and the other of R⁵ and R⁶ is hydrogen, then R⁴ is not —OCOR⁷ wherein R⁷ is heterocyclyl containing one nitrogen atom.

(b) Within the above embodiments (1), (2) and (3), including the subgroups contained therein, another group of compounds of Formula (I) is that wherein R³ is a ring of formula: 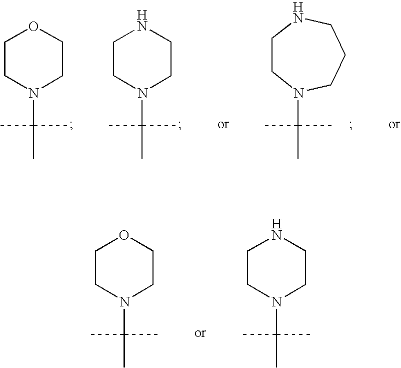
each substituted with R⁴, R⁵ and R⁶, including the hydrogen in —NH— groups in the rings, as defined in the Summary of the Invention. Within this embodiment (b), in one class of compounds R³ is morpholin-4-yl substituted with R⁴, R⁵ and R⁶, including the hydrogen in —NH— groups in the rings, as defined in the Summary of the Invention.

(c) Within the above embodiments (1), (2) and (3), including the subgroups contained therein, another group of compounds of Formula (I) is that wherein R³ is a ring of formula: 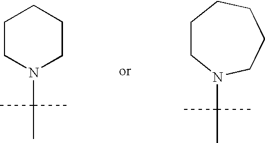
substituted with R⁴, R⁵ and R⁶, as defined in the Summary of the Invention provided that: (i) when R¹ and R² are hydrogen or alkyl, one of R⁵ and R⁶ is hydrogen or alkyl and the other of R⁵ and R⁶ is hydrogen, then R⁴ is not —NR⁸COR⁷ or —NR¹¹SO₂R⁷ (where R⁸ and R¹¹ are hydrogen, alkyl, hydroxyalkyl, alkoxyalkyl, aryl, heteroaryl, aralkyl, heteroaryl, heteroaralkyl or saturated monocyclic heterocyclylalkyl containing one nitrogen atom wherein the aryl or heteroaryl ring including the ring in aralkyl and heteroaralkyl are optionally substituted with alkyl, alkoxy, hydroxy, halo or acetamido and R⁷ is aryl, hetereoaryl, aralkyl, heteroaralkyl or saturated monocyclic heterocyclyl or heterocyclylalkyl containing one nitrogen atom and wherein the aryl or heteroaryl ring including the one in aralkyl and heteroaralkyl are optionally substituted with alkyl, alkoxy, hydroxy, halo or acetamido); (ii) when R¹ and R² are hydrogen or alkyl, R⁴ is hydrogen and one of R⁵ and R⁶ is hydrogen or alkyl, then the other of R⁵ and R⁶ is not —NRR′ [where R is hydrogen, alkyl, alkyl substituted with amino, monoalkyl or dialkylamino, hydroxyalkyl, alkoxyalkyl, aryl, heteroaryl, aralkyl, heteroaralkyl, or saturated monocyclic heterocyclylalkyl containing one nitrogen atom wherein the aryl or heteroaryl ring including the one in aralkyl and heteroaralkyl are optionally substituted with alkyl, alkoxy, hydroxy, halo or acetamido and R′ is —COR or —SO₂R (where R is alkyl, aryl, hetereoaryl, aralkyl, heteroaralkyl or saturated monocyclic heterocyclyl or heterocyclylalkyl containing one nitrogen atom and wherein the aryl or heteroaryl ring including the ring in aralkyl and heteroaralkyl are optionally substituted with alkyl, alkoxy, hydroxy, halo or acetamido)]; and (iii) when R¹ and R² are hydrogen or alkyl, one of R⁵ and R⁶ is hydrogen or alkyl and the other of R⁵ and R⁶ is hydrogen, then R⁴ is not —OCOR⁷ wherein R⁷ is heterocyclyl.

(d) Within the above embodiments (1), (2) and (3), including the subgroups contained therein, another group of compounds of Formula (I) is that wherein R³ is a ring of formula: 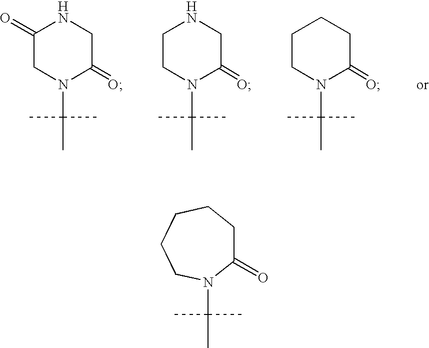
substituted with R⁴, R⁵ and R⁶, including the hydrogen in —NH— groups in the rings, as defined in the Summary of the Invention.

(e) Within the above embodiments (1), (2) and (3), including the subgroups contained therein, another group of compounds of Formula (I) is that wherein R³ is a ring of formula: 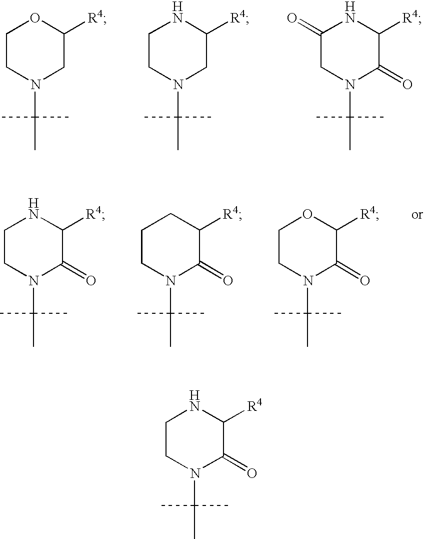
where R⁴ is cycloalkyl, cycloalkylalkyl, aryl, heteroaryl, heterocyclyl, aralkyl, heteroaralkyl, heterocyclylalkyl, or —XR⁷ (where X is —O—, —CO—, —C(O)O—, —OC(O)—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl) optionally substituted as defined in the Summary of the Invention and the rings are optionally substituted, including the hydrogen atom on the —NH— group within the ring with R⁵ and R as defined in the Summary of the Invention. In one group of compounds in this embodiment, R⁵ is hydrogen and R⁶ is on the carbon adjacent to the nitrogen attached to the quinazoline ring. In one group of compounds in this embodiment, R³ is morpholinyl or piperazinyl, R⁵ and R⁶ are hydrogen and R⁴ is phenyl substituted with R^{a}, R^{b} and R^{c} as defined in the Summary of the Invention provided that one of R^{a}, R^{b} and R^{c} is not hydrogen. In one group of compounds in this embodiment, R³ is morpholinyl or piperazinyl, R⁵ and R⁶ are hydrogen and R⁴ is —NHCOR⁷ where R⁷ is phenyl optionally substituted with R^{a}, R^{b} and R^{c} as defined in the Summary of the Invention provided that one of R^{a}, R^{b} and R^{c} is not hydrogen.

(f) Within the above embodiments (1), (2) and (3), including the subgroups contained therein, another group of compounds of Formula (I) is that wherein R³ is a ring of formula: 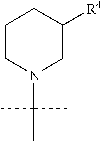
where R⁴ is cycloalkyl, cycloalkylalkyl, aryl, heteroaryl, aralkyl, heteroaralkyl, or —XR⁷ (where X is —O—, —CO—, —C(O)O—, —OC(O)—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl) optionally substituted as defined in the Summary of the Invention and the rings are optionally substituted, with R⁵ and R⁶ as defined in the Summary of the Invention. In one group of compounds in this embodiment, R⁵ is hydrogen and R⁶ is on the carbon adjacent to the nitrogen attached to the quinazoline ring. In one group of compounds in this embodiment, R⁵ and R⁶ are hydrogen and R⁴ is phenyl substituted with R^{a}, R^{b} and R^{c} as defined in the Summary of the Invention. In one group of compounds in this embodiment, R⁵ and R⁶ are hydrogen and R⁴ is —NHCOR⁷ where R⁷ is phenyl substituted with R^{a}, R^{b} and R^{c} as defined in the Summary of the Invention.

(g) Within the above embodiments (1), (2) and (3), including the subgroups contained therein, another group of compounds of Formula (I) is that wherein R³ is a ring of formula: 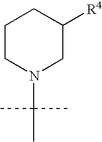
where R⁴ is heterocyclyl or heterocyclcylalkyl optionally substituted as defined in the Summary of the Invention and the rings are optionally substituted, with R⁵ and R⁶ as defined in the Summary of the Invention. In one group of compounds in this embodiment, R⁵ is hydrogen and R⁶ is on the carbon adjacent to the nitrogen attached to the quinazoline ring. In one group of compounds within this embodiment, R⁴ is a monocyclic heterocyclyl ring and substituted as described in (a). In another group of compounds within this embodiment, R⁴ is a monocyclic heterocyclyl containing only one or two heteroatoms and substituted as described in (a). In yet another group of compounds within this embodiment, R⁴ is a heterocyclyl ring fused to phenyl and substituted as described in (a). In another group of compounds within this embodiment, R⁴ is a heterocyclyl ring fused to phenyl containing only one or two heteroatoms and substituted as described in (a).

(h) Within the above embodiments (1), (2) and (3), including the subgroups contained therein, another group of compounds of Formula (I) is that wherein R³ is a ring of formula: 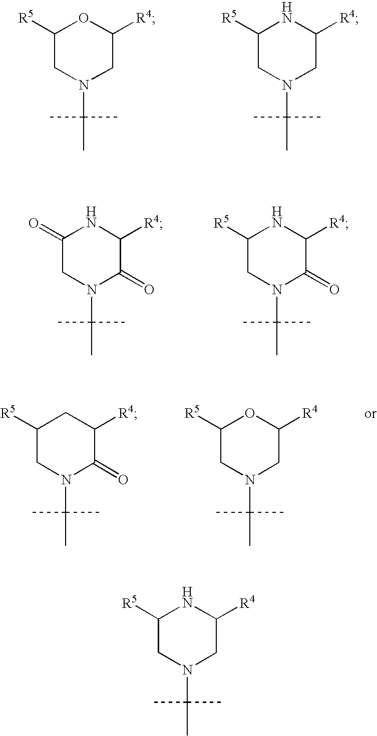
where R⁴ is phenyl or heteroaryl substituted at the para position with R^{a} and optionally substituted with R^{b} and R^{c} wherein R^{a}, R^{b}, and R^{c} are as defined in the Summary of the Invention and R⁵ is as defined in the Summary of the Invention and where the hydrogen atom on the —NH— group within the ring is optionally substituted with R⁵ or R⁶ as defined in the Summary of the Invention.

(i) Within the above embodiments (1), (2) and (3), including the subgroups contained therein, another group of compounds of Formula (I) is that wherein R³ is a ring of formula: 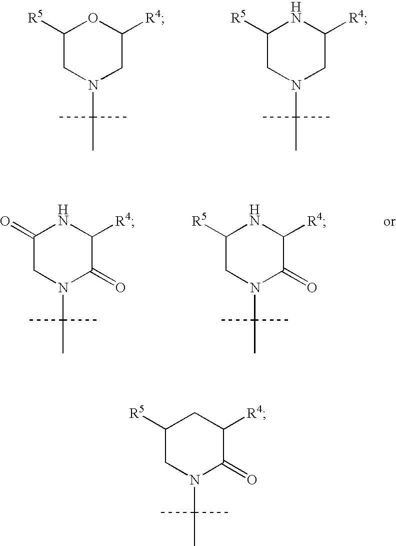
where R⁴ is cycloalkyl optionally substituted with one to three substitutents independently selected from R^{a}, R^{b}, and R^{c} where R^{a}, R^{b}, and R^{c} are as defined in the Summary of the Invention and R⁵ is as defined in the Summary of the Invention and where the hydrogen atom on the —NH— group within the ring is optionally substituted with R⁵ or R⁶ as defined in the Summary of the Invention.

(j) Within the above embodiments (1), (2) and (3), including the subgroups contained therein, another group of compounds of Formula (I) is that wherein R³ is a ring of formula: 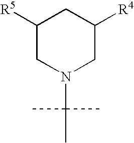
where R⁴ is phenyl or heteroaryl substituted at the para position with R^{a} and optionally substituted with R^{b} and R^{c} wherein R^{a}, R^{b}, and R^{c} are as defined in the Summary of the Invention and R⁵ is as defined in the Summary of the Invention.

(k) Within the above embodiments (1), (2) and (3), including the subgroups contained therein, another group of compounds of Formula (I) is that wherein R³ is a ring of formula: 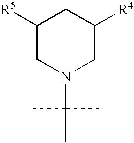
Where R⁴ is cycloalkyl optionally substituted with one to three substitutents independently selected from R^{a}, R^{b}, and R^{c} where R^{a}, R^{b}, and R^{c} are as defined in the Summary of the Invention and R⁵ is as defined in the Summary of the Invention.

(l) Within the above embodiments (1), (2) and (3), including the subgroups contained therein, another group of compounds of Formula (I) is that wherein R³ is a ring of formula: 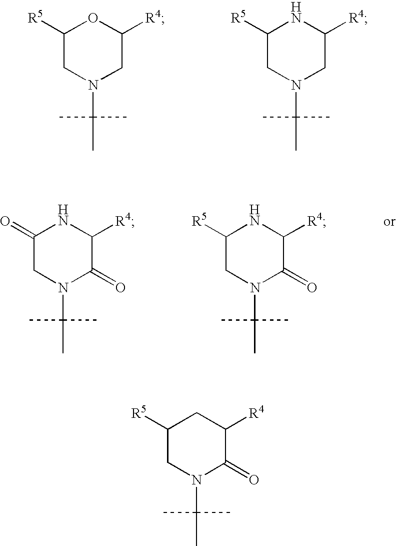
where R⁴ is heterocyclyl, preferably heterocyclyl containing at least a —C═O group wherein the heterocyclyl ring is optionally substituted at the para position with R^{a} and optionally substituted with R^{b} and R^{c} wherein R^{a}, R^{b}, and R^{c} are as defined in the Summary of the Invention and R⁵ is as defined in the Summary of the Invention. Within this group, in one embodiment, R⁴ is monocyclic saturated six membered ring containing at least a —C═O group and optionally substituted at the para position with R^{a} and optionally substituted with R^{b} and R^{c} wherein R^{a}, R^{b}, and R^{c} are as defined in the Summary of the Invention.

(m) Within the above embodiments (1), (2) and (3), including the subgroups contained therein, another group of compounds of Formula (I) is that wherein R³ is a ring of formula: 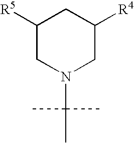
where R⁴ is heterocyclyl, preferably heterocyclyl containing at least a —C═O group wherein the heterocyclyl ring is optionally substituted at the para position with R^{a} and optionally substituted with R^{b} and R^{c} wherein R^{a}, R^{b}, and R^{c} are as defined in the Summary of the Invention and R⁵ is as defined in the Summary of the Invention. Within this group, in one embodiment, R⁴ is monocyclic saturated six membered ring containing at least a —C═O group and optionally substituted at the para position with R^{a} and optionally substituted with R^{b} and R^{c} wherein R^{a}, R^{b}, and R^{c} are as defined in the Summary of the Invention.

(n) Within the above embodiments (1), (2) and (3), including the subgroups contained therein, another group of compounds of Formula (I) is that wherein R³ is a ring of formula: 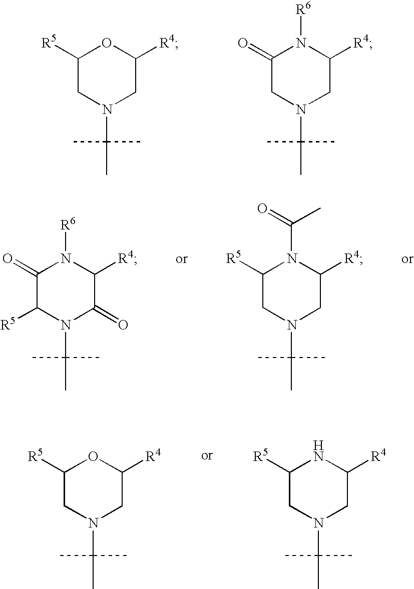
where R⁴ is cycloalkyl, phenyl, heteroaryl, or monocyclic saturated five or six membered heterocyclyl ring; R⁵ is hydrogen, alkyl, phenyl, heteroaryl, or monocyclic saturated five or six membered heterocyclyl ring; and R⁶ is alkyl, preferably methyl; and wherein the aromatic or alicyclic ring in R⁴ and R⁵ is optionally substituted with R^{a}, R^{b} and R^{c} as defined in the Summary of the Invention. Within this subgroup, in one embodiment R⁴ is phenyl, heteroaryl, or monocyclic saturated five or six membered heterocyclyl ring and R⁵ is hydrogen or alkyl. In another embodiment, R⁴ and R⁵ are independently phenyl, heteroaryl, or monocyclic saturated five or six membered heterocyclyl ring. In each of the above embodiments, the aromatic or alicyclic ring is optionally substituted with R^{a} selected from alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocylcyl and R^{b} and R^{c} independently selected from alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino. Within this subgroup, in one embodiment R⁴ is phenyl optionally substituted with R^{a}, R^{b} and R^{c} as defined in the Summary of the Invention provided that one of R^{a}, R^{b} and R^{c} is not hydrogen.

(o) Within the above embodiments (1), (2) and (3), including the subgroups contained therein, another group of compounds of Formula (I) is that wherein R³ is phenyl optionally substituted as defined in the Summary of the Invention.

Within this embodiment, one class of compounds is that wherein R³ is a group of formula: 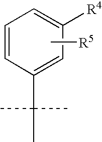
where one of R⁴ and R⁵ is hydrogen, alkyl, halo, haloalkyl, alkoxy, haloalkoxy, cyano, amino, monosubstituted or disubstituted amino, or —XR⁷ (where X is —O—, —CO—, —OC(O)—, —C(O)O, —NR⁸CO—, —CONR⁹—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); and the other of R⁴ and R⁵ is aryl, heteroaryl, or heterocyclyl; and wherein the aromatic or alicyclic ring in R⁴ and R⁵ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl.

In one embodiment within (o), R⁴ is aryl, heteroaryl, or heterocyclyl optionally substituted with one to three substitutents independently selected from R^{a}, R^{b}, and R^{c}. In another embodiment within (o), R⁴ is hydrogen, alkyl, or fluoro and R⁵ is heterocyclyl, monosubstituted or disubstituted amino, preferably R⁵ is located at the 4-position of the phenyl ring and the aromatic or alicyclic ring in R⁵ are optionally substituted with one to three substitutents independently selected from R^{a}, R^{b}, and R^{c}.

(p) Within the above embodiments (1), (2) and (3), including the subgroups contained therein, another group of compounds of Formula (I) is that wherein R³ is a ring of formula: 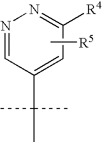
where R⁴ and R⁵ are as defined in (h) above.

(q) Within the above embodiments (1), (2) and (3), including the subgroups contained therein, another group of compounds of Formula (I) is that wherein R³ is a ring of formula: 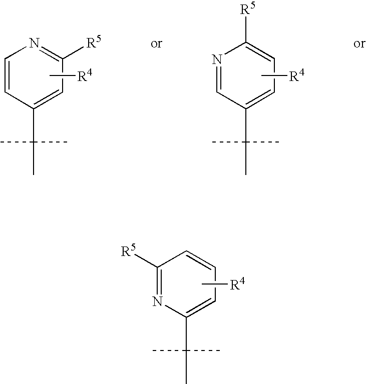
where R⁴ and R⁵ are as defined in (h) above.

In one embodiment, R³ is 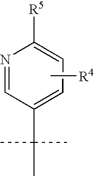
where R⁴ is hydrogen, alkyl, or fluoro and R⁵ is heterocyclyl, monosubstituted or disubstituted amino, preferably R⁴ is located at the 3-position of the pyridyl ring and the aromatic or alicyclic ring in R⁵ are optionally substituted with one to three substitutents independently selected from R^{a}, R^{b}, and R^{c}.

(r) Within the above embodiments (1), (2) and (3), including the subgroups contained therein, yet another group of compounds of Formula (I) is that wherein R³ is a ring of formula: 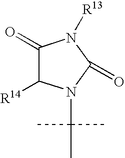
where R¹³ is aralkyl, preferably benzyl optionally substituted with R^{f}, R^{g} and R^{h} as defined in the Summary of the Invention and R¹⁴ is as defined in the Summary of the Invention, preferably hydrogen or alkyl.

(s) Within the above embodiments (1), (2), and (3), including the subgroups contained therein, yet another group of compounds of Formula (I) is that wherein R³ is pyrrolidin-1-yl substituted with R¹³, R¹⁴ and R¹⁵ as defined below.

R¹³ is cycloalkyl, aryl, heteroaryl, heterocyclyl, aralkyl, heteroaralkyl, heterocyclylalkyl, or —XR¹⁶ (where X is —O—, —CO—, —NR¹⁷CO—, —CONR¹⁶—, —NR¹⁹—, —S—, —SO—, —SO₂—, —NR²¹SO₂—, or —SO₂NR²¹— where R¹⁷-R²¹ are independently hydrogen, alkyl, hydroxyalkyl, alkoxyalkyl, aryl, aralkyl, heteroaryl, heteroaralkyl, acyl, or heterocyclylalkyl and R³⁴ is cycloalkyl, cycloalkylalkyl, aryl, heteroaryl, heterocyclyl, aralkyl, heteroaralkyl, or heterocyclylalkyl).

R¹⁴ is alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, aryl, heteroaryl or heterocyclyl.

R¹⁵ is hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino, preferably hydrogen.

The aromatic or alicyclic ring in R¹³, R¹⁴, R¹⁵, and R¹⁶ is optionally substituted with one to three substitutents independently selected from R^{r}, R^{g}, and R^{h} which are alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, sulfonyl, aminocarbonyl, aminosulfonyl, monosubstituted amino, disubstituted amino, optionally substituted phenyl, optionally substituted heteroaryl, or optionally substituted heterocyclyl; and additionally substituted with one or two substitutents independently selected from Rⁱ and R^{j} where Rⁱ and R^{j} are hydrogen or fluoro.

(t) Within the above embodiments (1), (2), and (3), including the subgroups contained therein, yet another group of compounds of Formula (I) is that wherein R³ is 2-oxopyrrolidinyl or 2,4-dioxoimidazolidinyl substituted with R³, R¹⁴ and R¹⁵ as defined below.

R¹³ is cycloalkyl, aryl, heteroaryl, heterocyclyl, aralkyl, heteroaralkyl, heterocyclylalkyl, or —XR¹⁶ (where X is —O—, —CO—, —NR¹⁷CO—, —CONR¹⁸—, —NR¹⁹—, —S—, —SO—, —SO₂—, —NR²¹SO₂—, or —SO₂NR²¹— where R¹⁷-R²¹ are independently hydrogen, alkyl, hydroxyalkyl, alkoxyalkyl, aryl, aralkyl, heteroaryl, heteroaralkyl, acyl, or heterocyclylalkyl and R¹⁶ is cycloalkyl, cycloalkylalkyl, aryl, heteroaryl, heterocyclyl, aralkyl, heteroaralkyl, or heterocyclylalkyl).

R¹⁴ is alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, aryl, heteroaryl or heterocyclyl.

R¹⁵ is hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino, preferably hydrogen.

The aromatic or alicyclic ring in R¹³, R¹⁴, R¹⁵, and R¹⁶ is optionally substituted with one to three substitutents independently selected from R^{f}, R^{g}, and R^{h} which are alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, sulfonyl, aminocarbonyl, aminosulfonyl, monosubstituted amino, disubstituted amino, optionally substituted phenyl, optionally substituted heteroaryl, or optionally substituted heterocyclyl; and additionally substituted with one or two substitutents independently selected from Rⁱ and R^{j} where Rⁱ and R^{j} are hydrogen or fluoro.

(u) Within the above embodiments (1), (2) and (3), including the subgroups contained therein, yet another group of compounds of Formula (I) is that wherein R³ is a ring of formula: 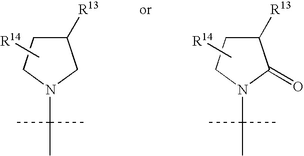
where R¹³ and R¹⁴ are as defined in the Summary of the Invention. Within this embodiment, one class of compounds of Formula (I) is that wherein R¹³ is cycloalkyl, aryl, heteroaryl, or heterocyclyl optionally substituted with one to three substitutents independently selected from R^{f}, R^{g}, and R^{h} and R¹⁴ is as defined in the Summary of the invention, preferably hydrogen or alkyl.

(v) Within the above embodiments (1), (2) and (3), including the subgroups contained therein, one group of compounds of Formula (I) is that wherein R³ is a ring of formula (a): 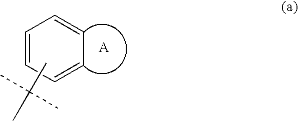
where A is a monocyclic five-, six-, or seven membered heterocyclyl ring substituted with R²², R²³ and R²⁴ as defined in the Summary of the Invention.

(w) Within the above embodiments (1), (2) and (3), including the subgroups contained therein, yet another group of compounds of Formula (I) is that wherein R³ is a ring of formula (a): 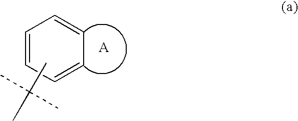
where A is a monocyclic five-, six-, or seven membered heterocyclyl ring and the ring (a) is substituted with R², R²³ and R²⁴ as defined below.

R²² is cycloalkyl, cycloalkylalkyl, aryl, heteroaryl, heterocyclyl, aralkyl, heteroaralkyl, heterocyclylalkyl, or —XR²⁵ (where X is —O—, —CO—, —NR²⁶CO—, —CONR²⁷—, —NR²⁸, —S—, —SO—, —SO₂—, —NR²⁹SO₂—, or —SO₂NR³⁰— where R²⁶-R³⁰ are independently hydrogen, alkyl, hydroxyalkyl, alkoxyalkyl, aryl, aralkyl, heteroaryl, heteroaralkyl, acyl, or heterocyclylalkyl and R²⁵ is cycloalkyl, cycloalkylalkyl, aryl, heteroaryl, heterocyclyl, aralkyl, heteroaralkyl, or heterocyclylalkyl).

R²³ is hydrogen, alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl.

R²⁴ is hydrogen, alkyl, alkoxy, halo, haloalkyl, haloalkoxy, hydroxyl, cyano, nitro, carboxy, alkoxycarbonyl, alkylthio, sulfinyl, sulfonyl, acyl, aminocarbonyl, aminosulfinyl, aminosulfonyl, or monosubstituted amino, disubstituted amino, preferably hydrogen.

The aromatic or alicyclic ring in R²², R²³, R²⁴, and R²⁵ is optionally substituted with one to three substitutents independently selected from R^{k}, R^{l}, and R^{m} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from Rⁿ and R^{o} where Rⁿ and R^{o} are hydrogen or fluoro.

(x) Within the above embodiments (1), (2) and (3), including the subgroups contained therein, another group of compounds of Formula (I) is that wherein R³ is a ring of formula: 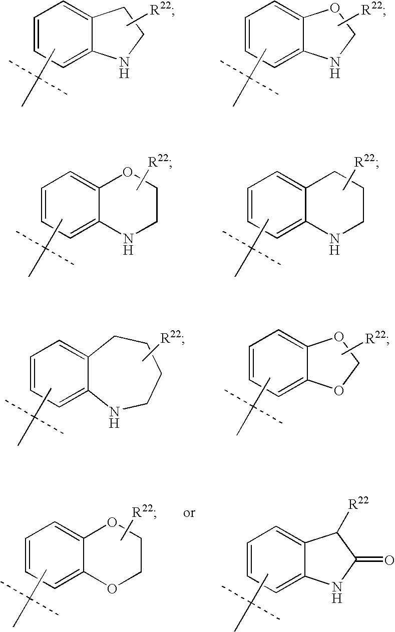
where R²² is as defined in the Summary of the Invention.

(y) Within the above embodiments (1), (2) and (3), including the subgroups contained therein, another group of compounds of Formula (I) is that wherein R³ is a ring of formula: 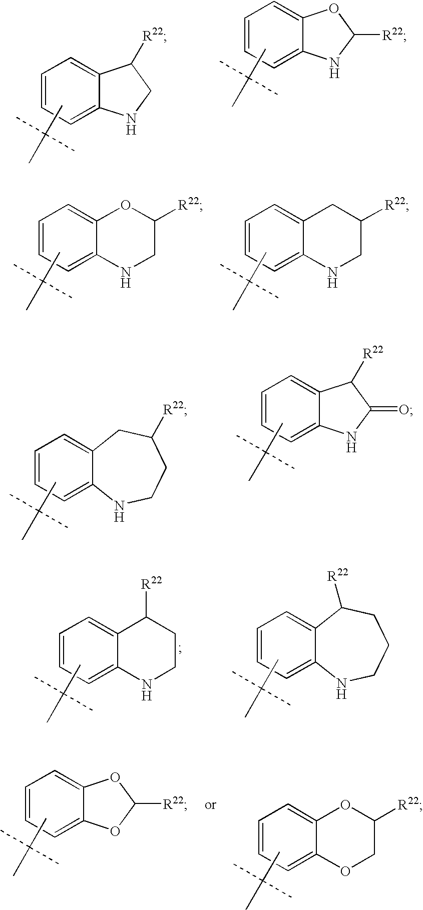
where R²² is as defined in the Summary of the Invention.

(z) Within the above embodiments (1), (2) and (3), including the subgroups contained therein, another group of compounds of Formula (I) is that wherein R³ is a ring of formula: 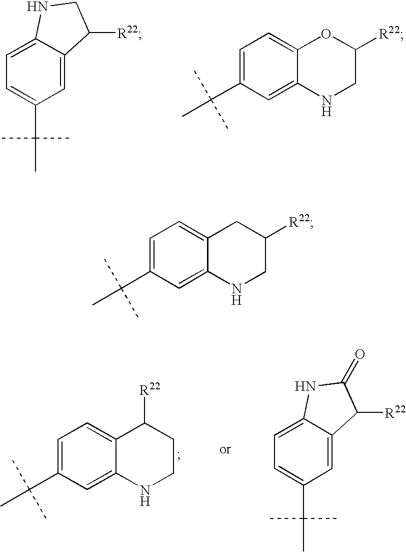
where R²² is as defined in the Summary of the Invention.

Within the subgroups (u)-(z) above, one group of compounds is that wherein R²² is phenyl optionally substituted as defined in the Summary of the Invention.

Within the subgroups (u)-(z) above, another group of compounds is that wherein R²² is heteroaryl optionally substituted as defined in the Summary of the Invention.

Within the subgroups (u)-(z) above, another group of compounds is that wherein R²² is a unsaturated monocyclic heterocyclyl optionally substituted as defined in the Summary of the Invention.

Within the subgroups (u)-(z) above, another group of compounds is that wherein R²² is saturated fused heterocyclyl optionally substituted as defined in the Summary of the Invention.

The R³ rings in subgroups (x)-(z) above, the subgroups contained therein, including the hydrogen in —NH— group in the rings, can also be optionally substituted with R²³ and R²⁴ are as defined in the Summary of the Invention. Preferably, one of R²³ and R²⁴ is hydrogen.

(aa) Within the above embodiments (1), (2) and (3), including the subgroups contained therein, yet another group of compounds of Formula (I) is that wherein R³ is a ring of formula (b) 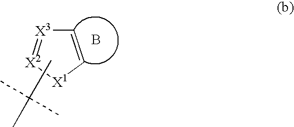
where X¹, X², and X³ are independently carbon, nitrogen, oxygen or sulfur provided that at least two of X¹, X², and X³ are other than carbon; and B is phenyl, or a six-membered heteroaryl ring (wherein the six-membered heteroaryl ring contains one or two nitrogen atoms, the rest of the ring atoms being carbon), or a monocyclic five-, six-, or seven-membered heterocyclyl ring; and wherein ring (b) is substituted with R³, R³² and R³³ as defined below.

R³¹ is cycloalkyl, cycloalkylalkyl, aryl, heteroaryl, heterocyclyl, aralkyl, heteroaralkyl, heterocyclylalkyl, or —XR³⁴ (where X is —O—, —CO—, —NR³⁵CO—, —CONR³⁶—, —NR³⁷—, —S—, —SO—, —SO₂—, —NR³⁸SO₂—, or —SO₂NR³⁹— where R³⁵-R³⁹ are independently hydrogen, alkyl, hydroxyalkyl, alkoxyalkyl, aryl, aralkyl, heteroaryl, heteroaralkyl, acyl, or heterocyclylalkyl and R³⁴ is cycloalkyl, cycloalkylalkyl, aryl, heteroaryl, heterocyclyl, aralkyl, heteroaralkyl, or heterocyclylalkyl).

R³² is hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl.

R³³ is hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino, preferably hydrogen.

The aromatic or alicyclic ring in R³¹, R³², R³³, and R³⁴ is optionally substituted with one to three substitutents independently selected from R^{p}, R^{q}, and R^{r} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{s} and R^{t} where R^{s} and R^{t} are hydrogen or fluoro.

In certain embodiments where R³ is a ring of formula (b), X¹ is carbon, nitrogen, oxygen or sulfur; and X² and X³ are each independently carbon or nitrogen, provided that at least two of X¹, X², and X³ are other than carbon. In some embodiments, X¹ is carbon and X² is nitrogen and X³ is nitrogen. In other embodiments, X¹ is nitrogen, X² is carbon and X³ is nitrogen. In yet other embodiments, X¹ is nitrogen, X² is nitrogen and X³ is carbon.

(ab) Within the above embodiments (1), (2) and (3), including the subgroups contained therein, another group of compounds of Formula (I) is that wherein R³ is a ring of formula (b). In one subgroup, R³ is a ring of formula: 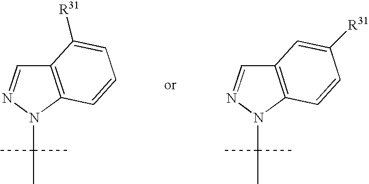
where R³¹ is cycloalkyl, cycloalkylalkyl, aryl, heteroaryl, heterocyclyl, aralkyl, heteroaralkyl, heterocyclylalkyl, or —XR³⁴ (where X is —O—, —CO—, —NR³⁵CO—, —CONR³⁶—, —NR³⁷—, —S—, —SO—, —SO₂—, —NR³⁸SO₂—, or —SO₂NR³⁹— where R³⁵-R³⁹ are independently hydrogen, alkyl, hydroxyalkyl, alkoxyalkyl, aryl, aralkyl, heteroaryl, heteroaralkyl, acyl, or heterocyclylalkyl and R¹⁶ is cycloalkyl, cycloalkylalkyl, aryl, heteroaryl, heterocyclyl, aralkyl, heteroaralkyl, or heterocyclylalkyl). In certain embodiments, R³¹ is phenyl, heteroaryl or heterocyclyl. Within group (ab), R³¹ is optionally substituted with R³² and R³³ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino. The aromatic or alicyclic ring in R³¹ is optionally substituted with one to three substitutents independently selected from R^{p}, R^{q}, and R^{r} which are alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino; and additionally substituted with one or two substitutents independently selected from R^{s} and R^{t} where R^{s} and R^{t} are hydrogen or fluoro.

In one group of compounds in (ab), R³ is: 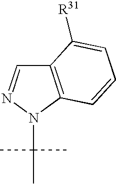
where R³¹ is morpholin-4-yl, piperazin-1-yl, or pyridinyl optionally substituted with one to three substituents independently selected from R^{p}, R^{q}, and R^{r} as defined in the Summary of the Invention.

(ac) Within the above embodiments (1), (2) and (3), including the subgroups contained therein, another group of compounds of Formula (I) is that wherein R³ is a ring of formula (c).

In some embodiments, a compound of Formula (I) as defined in the Summary of the Invention is provided with the proviso that the compound is not (a) monosubstituted piperazin-4-yl where the substituent is located at the N−1 nitrogen of the piperazine ring or homopiperazine ring; (b) disubstituted piperazinyl where one substituent is alkyl and the other is alkyl or —CONHR⁷ (where R⁷ is as defined in the Summary of the Invention); (c) trisubstituted piperazinyl where two substituents are alkyl and the third substituent is —CONHR⁷ (where R⁷ is as defined in the Summary of the Invention); (d) monosubstituted piperidin-1-yl where the substituent is located at the C-4 carbon of the piperidine ring; or (e) disubstituted piperidin-1-yl where one of the substituents is heterocyclyl and the other substituent is —OR⁷ (where R⁷ is as defined in the Summary of the Invention).

Representative compounds of Formula (I) are provided in Table 1 below.

TABLE 1

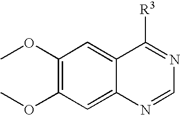

| Cpd. # | R³ |
|---|---|
| 1 | 7-(2-methoxyethoxy)-2,3-dihydro-1H-indol-1-yl |
| 2 | 4-morpholin-4-yl-2,3-dihydro-1H-indol-1-yl |
| 3 | 5-morpholin-4-yl-2,3-dihydro-1H-indol-1-yl |
| 4 | 2-(4-methoxyphenyl)morpholin-4-yl |
| 5 | 3-phenyl-1-(tert-butoxycarbonyl)piperazin-1-yl |
| 6 | 3-phenylpiperazin-1-yl |
| 7 | 3-(2-oxopiperidin-1-yl)piperidin-1-yl |
| 8 | 3-phenylpiperazin-1-yl |
| 9 | 4-(2-dimethylaminoethyl)-3-phenylpiperazin-1-yl |

General Synthetic Schemes

Compound of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromotography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compound of Formula (I) where R¹, R² and R³ are as defined in the Summary of the Invention can be prepared as described in Scheme 1 below.

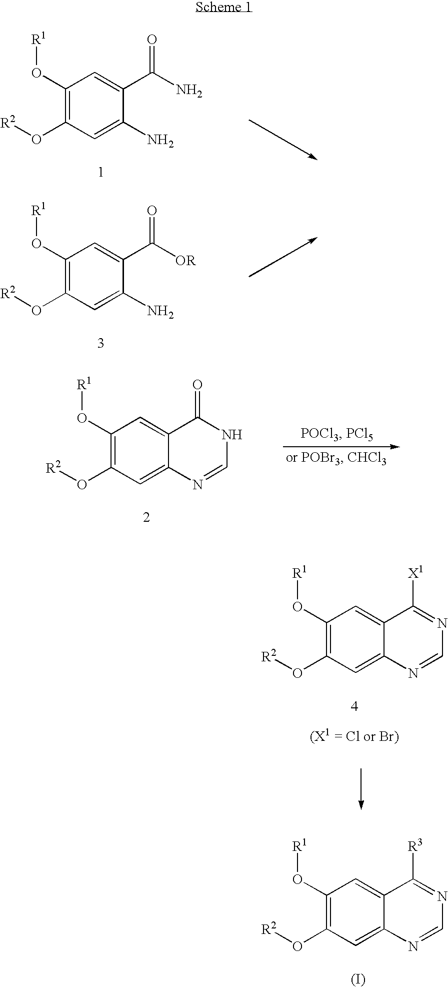

Reaction of 2-amino-4,5-dialkoxybenzamide of formula 1 with trimethyl orthoformate or 2-aminobenzoic ester compound of formula 3 with formamide in the presence of a base such as ammonium carbonate provides the corresponding 4-quinazolone 2 which upon treatment with either phosphorous oxychloride or phosphorous oxybromide provides the corresponding chloro or bromo compound of formula 4. The chloro derivative is prepared by heating 2 in neat phosphorous oxychloride, followed by recrystallization of the product after neutralization (see, for example, Castle et al., J. Org. Chem. 17:1571, 1952). The bromo derivative is prepared by mixing a concentrated suspension of the 4-hydroxycinnoline in chloroform and phosphorous oxybromide at room temperature and then warming to reflux for 8 to 16 h. Extractive workup after neutralization and subsequent recrystallization from alcoholic solvent such as ethanol provides 4-bromoquinazoline.

Compounds of formula 1 and 2 are either commercially available (e.g., methyl 2-amino-4,5-dimethoxybenzoate) or can be synthesized by methods common to the art. Simple dialkyl ethers, wherein the alkyl groups at the 3,4-postions are the same, can be readily accessed under standard etherification reactions. For example, 6,7-dimethoxy-4-quinazolone can be converted to 6,7-dihydroxy-4-quinazolone by treatment with BBr₃, which in turn can be treated with the desired alkyl halide in the presence of a base such as cesium carbonate, triethylamine, sodium hydride, potassium carbonate, potassium hydride, and the like to provide the dialkylated product. Suitable organic solvents include acetone, acetonitrile, DMF, THF, and the like.

Compounds of formula 2 where R¹ and R² are different can be prepared, by selectively protecting the 7-position in 6,7-dihydroxy-4-quinazolone as the benzyl ether (see Greenspan et al., J. Med. Chem. 42:164, 1999), converting the 6-hydroxy to the desired alkoxy group, followed by removal of the benzyl group at the 7-position, and alkylation of the resulting hydroxy group. Removal of the benzyl ether can be carried out under hydrogenolysis reaction conditions i.e., using palladium on carbon in alcoholic solvents such as methanol, ethanol and the like. This procedure can also be used to synthesize compounds of formula 3 where R¹ and R² are same or different from 3,4-dihydroxybenzoic esters. When R¹ and R² are different 3,4-dihydroxybenzoic esters is selectively benzylated at the 4-position, followed by alkylation of 3-hydroxy group with the desired alkyl group. Removal of the benzyl group, followed by alkyaltion provides 3,4-dialkoxybenzoic ester which is nitrated at the 6-position under standard nitration reaction conditions. Reduction of the nitro group then provides compound of formula 3.

If compounds of formula 1 where R¹ and R² are haloalkyl are desired, the corresponding hydroxy compound can be treated with haloacetic acid, e.g., chlorodifluoroacetic acid under basic conditions to provide difluoromethyl ether.

Compounds of Formula (I) wherein R³ is an aryl or heteroaryl ring, such as those shown in embodiments (O), (p), (q) and (v)-(z) above, can be prepared by standard synthetic methods known to one of ordinary skill in the art, for example, by Suzuki-type coupling of the corresponding aryl or heteroaryl boronic acid with 4-chloro-quinazoline 4 (see, e.g., Miyaura and Suzuki, Chem. Rev. 95:2457-2483, 1995). Such boronic acids are either commercially available (e.g., Aldrich Chemical Co. (Milwaukee, Wis.), Lancaster Synthesis (Ward Hill, Mass.), or Maybridge (Conrwall, UK)) or can readily be prepared from the corresponding bromides by methods described in the literature (see, for example, N. Miyaura et al, Tetrahedron Letters 1979, 3437; N. Miyaura, A. Suzuki, Chem. Commun. 1979, 866).

Compounds of Formula (I) where R³ is heterocyclic ring attached to the quinazoline ring via a nitrogen atom e.g., pyrrolidin-1-yl, piperidin-1-yl, morpolin-4-yl, and the like (see, for example, embodiments (b)-(n) and (r)-(u) above), are prepared by reacting 4 with the heterocyclic ring where X¹ is halo or other suitable leaving group such as tosylate, triflate, mesylate and the like in the presence of a base such as triethylamine, pyridine, and the like. Suitable solvents include, and the not limited to, tetrahydrofuran, DMF, and the like. Such heterocyclic rings (pyrrolidines, piperidines, homopiperidines, piperazines, homopiperazines, morpholines, and the like) are either commercially available or can be readily prepared by standard methods known within the art (see, e.g., Louie and Hartwig, Tetrahedron Letters 36:3609, 1995; Guram et al., Angew Chem. Int. Ed. 34:1348, 1995). Alternatively, a compound of Formula (I) is prepared by heating 4 with the heterocyclic ring in a suitable organic solvent such as THF, benzene, dioxane, toluene, alcohol, or mixtures thereof, under catalytic conditions using, for example, a palladium or copper catalyst (such as, but not limited to tris(dibenzylideneacetone) dipalladium(o) or copper (I) iodide) in the presence of a suitable base such as potassium carbonate, sodium t-butoxide, lithium hexamethyldisilizane, and the like.

Substituted indazoles useful to make compounds of Formula (I) wherein R³ is a ring as shown in embodiment z above are either commercially available (e.g., Aldrich Chemical Co., Sinova, Inc. (Bethesda, Mass.), J & W PharmLab, LLC (Morrisville, Pa.)) or can be prepared by methods commonly known within the art (see, e.g., Lebedev et al., J. Org. Chem. 70(2):596-602, 2005, and the references cited therein). For example, indazoles wherein R³¹ is heterocyclyl, for example, morpholine or N-methylpiperazine, may be synthesized by Buchwald-type coupling of the corresponding bromoindazole with the desired heterocyclic compound. The bromoindazoles can be prepared as described in International Publication No. WO 2004/029050, the disclosure of which is incorporated herein by reference in its entirety. Copper catalyzed reaction of the appropriately substituted indazole with 4 provides the appropriate compound of Formula (I). Alternatively, the bromoindazole undergoes palladium catalyzed reaction with 4 to provide 6,7-dimethoxy-4-(bromo-1H-indazol-1-yl)quinazoline. Subsequent N-arylation reaction with, for example morpholine or N-methylpiperazine provides the desired compound of Formula (I). Alternatively, Suzuki-type reaction of 6,7-dimethoxy-4-(bromo-1H-indazol-1-yl)quinazoline with aryl or heteroaryl boronic acids, for example, phenylboronic acid or 4-pyridine boronic acid, gives the corresponding aryl or heteroaryl substituted indazole quinazoline of Formula (I).

Utility and Methods of Use

In one aspect, methods are provided for treating a disorder or disease treatable by inhibition of PDE10 comprising administering a therapeutically effective amount of compound as provided herein to a patient in need thereof to treat the disorder or disease.

The compounds of the present invention inhibit PDE10 enzyme activity and hence raise the levels of cAMP or cGMP within cells that express PDE10. Accordingly, inhibition of PDE10 enzyme activity can be useful in the treatment of diseases caused by deficient amounts of cAMP or cGMP in cells. PDE10 inhibitors can also be of benefit in cases wherein raising the amount of cAMP or cGMP above normal levels results in a therapeutic effect. Inhibitors of PDE10 can be used to treat disorders of the peripheral and central nervous system, cardiovascular diseases, cancer, gastro-enterological diseases, endocrinological diseases and urological diseases.

Indications that may be treated with PDE10 inhibitors, either alone or in combination with other drugs, include, but are not limited to, those diseases thought to be mediated in part by the basal ganglia, prefrontal cortex and hippocampus. These indications include psychoses, Parkinson's disease, dementias, obsessive compulsive disorder, tardive dyskinesia, choreas, depression, mood disorders, impulsivity, drug addiction, attention deficit/hyperactivity disorder (ADHD), depression with parkinsonian states, personality changes with caudate or putamen disease, dementia and mania with caudate and pallidal diseases, and compulsions with pallidal disease.

Psychoses are disorders that affect an individual's perception of reality. Psychoses are characterized by delusions and hallucinations. The compounds of the present invention would be useful in treating patients suffering from all forms of psychoses, including, but not limited to, schizophrenia, late-onset schizophrenia, schizoaffective disorders, prodromal schizophrenia, and bipolar disorders. Treatment can be for the positive symptoms of schizophrenia as well as for the cognitive deficits and negative symptoms. Other indications for PDE10 inhibitors include psychoses resulting from drug abuse (including amphetamines and PCP), encephalitis, alcoholism, epilepsy, Lupus, sarcoidosis, brain tumors, multiple sclerosis, dementia with Lewy bodies, or hypoglycemia. Other psychiatric disorders, like posttraumatic stress disorder (PTSD), and schizoid personality can also be treated with PDE10 inhibitors.

Obsessive-compulsive disorder (OCD) has been linked to deficits in the frontal-striatal neuronal pathways (Saxena et al., Br. J. Psychiatry Suppl, 35:26-37, 1998). Neurons in these pathways project to striatal neurons that express PDE10. PDE10 inhibitors cause cAMP to be elevated in these neurons; elevations in cAMP result in an increase in CREB phosphorylation and thereby improve the functional state of these neurons. The compounds of the present invention are therefore suitable for use in the indication of OCD. OCD may result, in some cases, from streptococcal infections that cause autoimmune reactions in the basal ganglia (Giedd et al., Am J Psychiatry. 157:281-283, 2000). Because PDE10 inhibitors may serve a neuroprotective role, administration of PDE10 inhibitors may prevent the damage to the basal ganglia after repeated streptococcal infections and thereby prevent the development of OCD.

In the brain, the level of cAMP or cGMP within neurons is believed to be related to the quality of memory, especially long term memory. Without wishing to be bound to any particular mechanism, it is proposed that since PDE10 degrades cAMP or cGMP, the level of this enzyme affects memory in animals, for example, in humans. For example, a compound that inhibits cAMP phosphodiesterase (PDE) can thereby increase intracellular levels of cAMP, which in turn activate a protein kinase that phosphorylates a transcription factor (cAMP response binding protein), which transcription factor then binds to a DNA promoter sequence to activate genes that are important in long term memory. The more active such genes are, the better is long-term memory. Thus, by inhibiting a phosphodiesterase, long term memory can be enhanced.

Dementias are diseases that include memory loss and additional intellectual impairment separate from memory. The compounds of the present invention can be useful for treating patients suffering from memory impairment in all forms of dementia. Dementias are classified according to their cause and include: neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, bacterial meningitis, Creutzfeld-Jacob Disease, multiple sclerosis, traumatic (e.g., subdural hematoma or traumatic brain injury), infectious (e.g., HIV), genetic (down syndrome), toxic (e.g., heavy metals, alcohol, some medications), metabolic (e.g., vitamin B12 or folate deficiency), CNS hypoxia, Cushing's disease, psychiatric (e.g., depression and schizophrenia), and hydrocephalus.

The condition of memory impairment is manifested by impairment of the ability to learn new information and/or the inability to recall previously learned information. The present invention includes methods for dealing with memory loss separate from dementia, including mild cognitive impairment (MCI) and age-related cognitive decline. The present invention includes methods of treatment for memory impairment as a result of disease. Memory impairment is a primary symptom of dementia and can also be a symptom associated with such diseases as Alzheimer's disease, schizophrenia, Parkinson's disease, Huntington's disease, Pick's disease, Creutzfeld-Jakob disease, HIV, cardiovascular disease, and head trauma as well as age-related cognitive decline. The compounds of the present invention would be useful in the treatment of memory impairment due to, for example, Alzheimer's disease, multiple sclerosis, amylolaterosclerosis (ALS), multiple systems atrophy (MSA), schizophrenia, Parkinson's disease, Huntington's disease, Pick's disease, Creutzfeld-Jakob disease, depression, aging, head trauma, stroke, spinal cord injury, CNS hypoxia, cerebral senility, diabetes associated cognitive impairment, memory deficits from early exposure of anesthetic agents, multiinfarct dementia and other neurological conditions including acute neuronal diseases, as well as HIV and cardiovascular diseases.

The compounds of the present invention are also suitable for use in the treatment of a class of disorders known as polyglutamine-repeat diseases. These diseases share a common pathogenic mutation. The expansion of a CAG repeat, which encodes the amino acid glutamine, within the genome leads to production of a mutant protein having an expanded polyglutamine region. For example, Huntington's disease has been linked to a mutation of the protein huntingtin. In individuals who do not have Huntington's disease, huntingtin has a polyglutamine region containing about 8 to 31 glutamine residues. For individuals who have Huntington's disease, huntingtin has a polyglutamine region with over 37 glutamine residues. Aside from Huntington's disease (HD), other known polyglutamine-repeat diseases and the associated proteins include dentatorubral-pallidoluysian atrophy, DRPLA (atrophin-1); spinocerebellar ataxia type-1 (ataxin-1); spinocerebellar ataxia type-2 (ataxin-2); spinocerebellar ataxia type-3 also called Machado-Joseph disease, MJD (ataxin-3); spinocerebellar ataxia type-6 (alpha 1a-voltage dependent calcium channel); spinocerebellar ataxia type-7 (ataxin-7); and spinal and bulbar muscular atrophy, SBMA, also know as Kennedy disease (androgen receptor).

The basal ganglia are important for regulating the function of motor neurons; disorders of the basal ganglia result in movement disorders. Most prominent among the movement disorders related to basal ganglia function is Parkinson's disease (Obeso et al., Neurology. 62(1 Suppl 1):S17-30, 2004). Other movement disorders related to dysfunction of the basla ganglia include tardive dyskinesia, progressive supranuclear palsy and cerebral palsy, corticobasal degeneration, multiple system atrophy, Wilson disease, and dystonia, tics, and chorea. The compounds of the invention can be used to treat movement disorders related to dysfunction of basal ganglia neurons.

PDE10 inhibitors can be used to raise cAMP or cGMP levels and prevent neurons from undergoing apoptosis. PDE10 inhibitors may be anti-inflammatory by raising cAMP in glial cells. The combination of anti-apoptotic and anti-inflammatory properties, as well as positive effects on synaptic plasticity and neurogenesis, make these compounds useful to treat neurodegeneration resulting from any disease or injury, including stroke, spinal cord injury, Alzheimer's disease, multiple sclerosis, amylolaterosclerosis (ALS), and multiple systems atrophy (MSA).

Autoimmune diseases or infectious diseases that affect the basal ganglia may result in disorders of the basal ganglia including ADHD, OCD, tics, Tourette's disease, Sydenham chorea. In addition, any insult to the brain can potentially damage the basal ganglia including strokes, metabolic abnormalities, liver disease, multiple sclerosis, infections, tumors, drug overdoses or side effects, and head trauma. Accordingly, the compounds of the invention can be used to stop disease progression or restore damaged circuits in the brain by a combination of effects including increased synaptic plasticity, neurogenesis, anti-inflammatory, nerve cell regeneration and decreased apoptosis

The growth of some cancer cells is inhibited by cAMP and cGMP. Upon transformation, cells may become cancerous by expressing PDE10 and reducing the amount of cAMP or cGMP within cells. In these types of cancer cells, inhibition of PDE10 activity will inhibit cell growth by raising cAMP. In some cases, PDE10 may be expressed in the transformed, cancerous cell but not in the parent cell line. In transformed renal carcinoma cells, PDE1 is expressed and PDE10 inhibitors reduce the growth rate of the cells in culture. Similarly, breast cancer cells are inhibited by administration of PDE10 inhibitors. Many other types of cancer cells may also be sensitive to growth arrest by inhibition of PDE10. Therefore, compounds disclosed in this invention can be used to stop the growth of cancer cells that express PDE10.

The compounds of the invention are also suitable for use in the treatment of diabetes and related disorders such as obesity, by focusing on regulation of the cAMP signaling system. By inhibiting PDE-10A activity, intracellular levels of cAMP and increased, thereby increasing the release of insulin-containing secretory granules and, therefore, increasing insulin secretion. See, for example, WO 2005/012485, which is hereby incorporated by reference in its entirety. The compounds of Formula (I) can also be used to treat the diseases disclosed in US Patent application publication No. 2006/019975, the disclosure of which is incorporated herein by reference in its entirety.

Testing

The PDE10 inhibitory activities of the compounds of the present invention can be tested using the in vitro and in vivo assays described in the Examples below.

Administration and Pharmaceutical Compositions

In general, the compounds of this invention can be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. Therapeutically effective amounts of compounds of formula (I) may range from approximately 0.1-1000 mg per day; preferably 0.5 to 250 mg/day, more preferably 3.5 mg to 70 mg per day.

In general, compounds of this invention can be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of formula (I) in combination with at least one pharmaceutically acceptable excipient. Acceptable excipients are non-toxic, aid administration, and do not adversely affect the therapeutic benefit of the compound of formula (I). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 18th ed., 1990).

The level of the compound in a formulation can vary within the full range employed by those skilled in the art. Typically, the formulation will contain, on a weight percent (wt %) basis, from about 0.01-99.99 wt % of a compound of formula (I) based on the total formulation, with the balance being one or more suitable pharmaceutical excipients. Preferably, the compound is present at a level of about 1-80 wt %.

The compounds can be administered as the sole active agent or in combination with other pharmaceutical agents such as other agents used in the treatment of psychoses, especially schizophrenia and bipolar disorder, obsessive-compulsive disorder, Parkinson's disease, Alzheimer's disease, cognitive impairment and/or memory loss, e.g., nicotinic α-7 agonists, PDE4 inhibitors, other PDE10 inhibitors, calcium channel blockers, muscarinic m1 and m2 modulators, adenosine receptor modulators, ampakines, NMDA-R modulators, mGluR modulators, dopamine modulators, serotonin modulators, canabinoid modulators, and cholinesterase inhibitors (e.g., donepezil, rivastigimine, and galanthanamine). In such combinations, each active ingredient can be administered either in accordance with their usual dosage range or a dose below their usual dosage range and can be administered either simultaneously or sequentially.

Drugs suitable in combination with the compounds of the present invention include, but not limited to, other suitable schizophrenia drugs such as Clozaril, Zyprexa, Risperidone, and Seroquel; bipolar disorder drugs such as Lithium, Zyprexa, and Depakote, Parkinson's disease drugs such as Levodopa, Parlodel, Permax, Mirapex, Tasmar, Contan, Kemadin, Artane, and Cogentin; agents used in the treatment of Alzheimer's disease such as, but not limited to, Reminyl, Cognex, Aricept, Exelon, Akatinol, Neotropin, Eldepryl, Estrogen and Cliquinol; agents used in the treatment of dementia such as, but not limited to, Thioridazine, Haloperidol, Risperidone, Cognex, Aricept, and Exelon; agents used in the treatment of epilepsy such as, but not limited to, Dilantin, Luminol, Tegretol, Depakote, Depakene, Zarontin, Neurontin, Barbita, Solfeton, and Felbatol; agents used in the treatment of multiple sclerosis such as, but not limited to, Detrol, Ditropan XL, OxyContin, Betaseron, Avonex, Azothioprine, Methotrexate, and Copaxone; agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone; agents useful in the treatment of diabetes, including, but not limited to, PPAR ligands (e.g. agonists, antagonists, such as Rosiglitazone, Troglitazone and Pioglitazone), insulin secretagogues (for example, sulfonylurea drugs (such as Glyburide, Glimepiride, Chlorpropamide, Tolbutamide, and Glipizide) and non-sulfonyl secretagogues), α-glucosidase inhibitors (such as Acarbose, Miglitol, and Voglibose), insulin sensitizers (such as the PPAR-γ agonists, e.g., the glitazones; biguanides, PTP-1B inhibitors, DPP-IV inhibitors and 11beta-HSD inhibitors), hepatic glucose output lowering compounds (such as glucagon antagonists and metaformin, such as Glucophage and Glucophage XR), insulin and insulin derivatives (both long and short acting forms and formulations of insulin), and anti-obesity drugs, including but not limited to β-3 agonists, CB-1 agonists, neuropeptide Y5 inhibitors, Ciliary Neurotrophic Factor and derivatives (e.g., Axokine), appetite suppressants (e.g., Sibutramine), and lipase inhibitors (e.g., Orlistat).

### EXAMPLES

The following preparations and examples are given to enable those skilled in the art to more clearly understand and to practice the present invention. They should not be considered as limiting the scope of the invention, but merely as being illustrative and representative thereof. All spectra were recorded at 300 MHz on a Bruker Instruments NMR unless otherwise stated. Coupling constants (J) are in Hertz (Hz) and peaks are listed relative to TMS (δ0.00 ppm). Microwave reactions were performed using a Personal Chemistry Optimizer™ microwave reactor in 10 mL Personal Chemistry microwave reactor vials. All reactions were performed at 200° C. for 600 s with the fixed hold time ON unless otherwise stated. Sulfonic acid ion exchange resins (SCX) were purchased from Varian Technologies. Analytical HPLC was performed on 4.6 mm×100 mm Waters Sunfire RP C18 5 μm column using (i) a gradient of 20/80 to 80/20 acetonitrile (0.1% formic acid)/water (0.1% formic acid) over 6 min (Method A), (ii) a gradient of 20/80 to 80/20 acetonitrile (0.1% formic acid)/water (0.1% formic acid) over 8 min (Method B), (iii) a gradient of 40/60 to 80/20 acetonitrile (0.1% formic acid)/water (0.1% formic acid) over 6 min (Method C), or (iv) a gradient of 40/60 to 80/420 acetonitrile (0.1% formic acid)/water (0.1% formic acid) over 8 min (Method D). Preparative HPLC was performed on 30 mm×100 mm Xtera Prep RP₁₈ 5μ columns using an 8 min gradient of 95/5 to 20/80 water (0.1% formic acid)/acetonitrile (0.1% formic acid).

### Synthetic Examples

### Example 1

### Synthesis of 6,7-dimethoxy-4-[7-(2-methoxyethoxy)-2,3-dihydro-1H-indol-1-yl]quinazoline

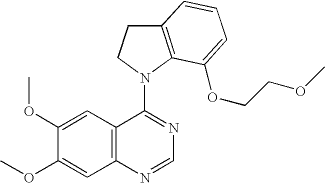

4-Chloro-6,7-dimethoxyquiinazoline (111 mg, 0.494 mmol) was added to a solution of 7-(2-methoxyethoxy)indoline (95.5 mg, 0.494 mmol) in N,N-dimethylacetamide (5.0 mL). Sodium iodide (30 mg, 0.2 mmol) and potassium carbonate (273 mg, 1.98 mmol) were then added, and the resulting mixture was heated at 160° C. for 12 h. The crude mixture was filtered through an SCX column (using a solution of 2N ammonia in methanol as eluent). Volatiles were removed by evaporation, and the residue was purified by preparative HPLC (using a 10:90 to 80:20 gradient of acetonitrile:water (with 0.1% formic acid) and a flow rate of 45 mL/min). Further purification using a Berger SFC Minigram instrument (using 15% methanol modifier on a pyridine column at a pressure of 120 bar and a flow rate of 9.9 mL/min and a column temperature of 35° C.) afforded 3.2 mg (1.7% yield) of 6,7-dimethoxy-4-[7-(2-methoxyethoxy)-2,3-dihydro-1H-indol-1-yl]quinazoline. ¹H NMR (CDCl₃) δ 8.70 (s, 1H), 7.27 (s, 1H), 7.13 (s, 1H), 6.97 (m, 2H), 6.81 (d, J=7.1, 1H), 4.40 (m, 2H), 4.05 (s, 3H), 4.00 (m, 2H), 3.84 (s, 3H), 3.22 (m, 2H), 3.13 (m, 2H), 3.02 (s, 3H), LC/MS (EI) t_{R} 3.69 min (Method B), m/z 382 (M⁺+1).

### Example 2

### Synthesis of 6,7-dimethoxy-4-(4-morpholin-4-yl-2,3-dihydro-1H-indol-1-yl)quinazoline

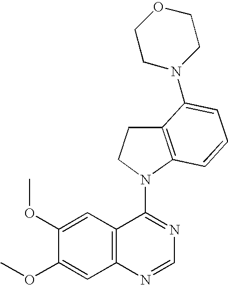

Step 1. 4-Bromoindole (5.00 mL, 39.9 mmol) was dissolved in a mixture of acetic acid (5.00 mL, 87.9 mmol) and methanol (25.0 mL, 617 mmol) and cooled to 0° C. Sodium cyanoborohydride (7.52 g, 0.120 mol) was added and the mixture was slowly warmed to room temperature over a period of 1 h. The reaction mixture was then concentrated and neutralized using a saturated aqueous solution of sodium bicarbonate. The organics were extracted with ether and ethyl acetate (and the combined organics were washed with brine, dried, filtered, and concentrated to afford 4.11 g (52% yield) of 4-bromoindoline.

Step 2. 4-Chloro-6,7-dimethoxyquinazoline (2.20 g, 9.9 mmol) was added to a solution of 4-bromoindoline (1.97 g, 9.95 mmol, prepared as described in Step 1) in N,N-dimethylacetamide (50 mL). Sodium iodide (0.7 g, 4 mmol) and potassium carbonate (0.55 g, 39.8 mmol) were then added, and the resulting mixture was heated at 160° C. for 2.75 h. The reaction mixture was diluted with water and extracted with ethyl acetate (100 mL). The organic layer was washed with water and brine, dried, filtered, and concentrated to afford 1.89 g of 4-(4-bromo-2,3-dihydro-1H-indol-1-yl)-6,7-dimethoxyquinazoline.

Step 3. 4-(4-Bromo-2,3-dihydro-1H-indol-1-yl)-6,7-dimethoxyquinazoline (0.2 g, 0.5 mmol, prepared as described above in Step 2), morpholine (54.2 μL, 0.621 mmol) tetrahydrofuran (4.00 mL), tris(dibenzylideneacetone)dipalladium(0) (20 mg, 0.02 mmol), 9,9-dimethyl-4,5-bis(diphenylphosphino)xanthane (30 mg, 0.052 mmol), sodium tert-butoxide (74.6 mg, 0.777 mmol) were added to a 10 ml sealed microwave tube and the resulting mixture was heated to 50° C. for 8 h. The mixture was loaded onto an SCX column, which was washed with 1 column volume of methanol. The product was eluted using ammonia in methanol and the organics were concentrated to afford 3 mg of 6,7-dimethoxy-4-(4-morpholin-4-yl-2,3-dihydro-1H-indol-1-yl)quinazoline. ¹H NMR (CDCl₃) δ 8.78 (s, 1H), 7.30 (s, 1H), 7.27 (s, 1H), 7.02 (t, J=8.0, 1H), 6.51 (m, 2H), 4.35 (m, 2H), 4.07 (s, 3H), 3.89 (m, 4H), 3.50 (s, 3H), 3.14 (m, 2H), 3.09 (m, 4H), LC/MS (EI) t_{R} 3.79 min (Method B), m/z 393 (M⁺+1).

### Example 3

### Synthesis of 6,7-dimethoxy-4-(5-morpholin-4-yl-2,3-dihydro-1H-indol-1-yl)quinazoline hydroformate

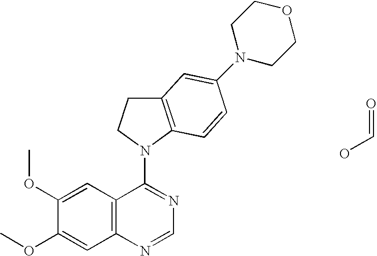

5-bromoindoline was prepared by following the procedure in Step 1 of Example 2 in which 5-bromoindole was used in place of 4-bromoindole, and used in Step 2 of Example 2 to prepare the title compound. The product was further purified buy column chromatography (using a gradient of 10:90 to 80:20 acetonitrile:water (with 1% formic acid). 49 mg (19% yield in Step 3). ¹H NMR (CDCl₃) δ 8.70 (s, 1H), 8.38 (s, 1H), 7.46 (s, 1H), 7.06 (m, 1H), 6.92 (s, 1H), 6.81 (s, 1H), 6.68 (m, 1H), 4.40 (m, 2H), 4.06 (s, 3H), 3.85 (m, 4H), 3.84 (s, 3H), 3.20 (m, 2H), 3.11 (m, 4H) LC/MS (EI) t_{R} 3.82 min (Method B), m/z 393 (M⁺+1).

### Example 4

### Synthesis of 6,7-dimethoxy-4-(3-phenylpiperazin-1-yl)quinazoline

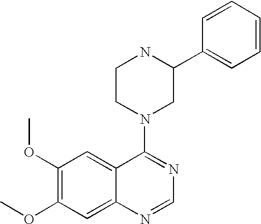

Step 1. 4-Chloro-6,7-dimethoxyquinazoline (856 mg, 3.81 mmol), tert-butyl 2-phenylpiperazine-1-carboxylate (1.0 g, 3.81 mmol), N,N-dimethylacetamide (15 mL), tetra-n-butylammonium iodide (140 mg, 0.38 mmol) and potassium carbonate (1.58 g, 11.4 mmol) were combined and warmed to 100° C. for 3 hours, concentrated under vacuum at 55° C. and the residue was dissolved in 100 mL of water and 200 mL of DCM. The organic phase was separated, dried (MgSO₄), concentrated and purified by column chromatography over silica gel using a gradient elution going from 0% MeOH to 5% MeOH in 1:1 EtOAc/hexane with 0.3% DMEA to provide tert-butyl 4-(6,7-dimethoxyquinazolin-4-yl)-2-phenylpiperazine-1-carboxylate as a white solid.

Step 2. Tert-Butyl 4-(6,7-dimethoxyquinazolin-4-yl)-2-phenylpiperazine-1-carboxylate (1.36 g, 3.02 mmol), trifluoroacetic acid (5.0 mL) and DCM (10 mL) were combined and stirred at room temperature for 5 hours. The reaction mixture was concentrated and the residue was triturated to give 6,7-dimethoxy-4-(3-phenylpiperazin-1-yl)quinazoline trifluoroacetate as a white solid which was directly used for next reaction. The trifluoroacetic acid salt was dissolved in 150 mL of DCM and washed with 0.5 M NaOH (20 mL). The organic phase was separated, dried (MgSO₄) and absorbed onto silica gel and purified by column chromatography using 5% MeOH in DCM as eluant to provide 6,7-dimethoxy-4-(3-phenylpiperazin-1-yl)quinazoline as a white solid. ¹H NMR (CDCl₃) δ 8.69 (s, 1H), 7.58-7.43 (m, 6H), 7.09 (s, 1H), 4.74 (t, 2H), 4.42-4.21 (m, 3H) 4.06 (s, 3H), 3.95 (s, 3H), 3.58-3.55 (m, 1H), 3.36-3.33 (m, 2H). LCMS: Retention time=2.98 minutes, M+H=351.

### Example 5

### Synthesis of 2-[4-(6,7-Dimethoxyquinazolin-4-yl)-2-phenylpiperazin-1-yl]-N,N-dimethylethanamine

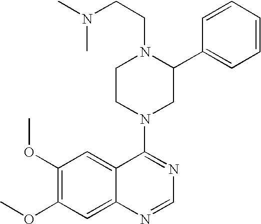

6,7-Dimethoxy-4-(3-phenylpiperazin-1-yl)quinazoline (30 mg, 0.086 mmol), β-dimethylaminoethylchloride hydrochloride (16 mg, 0.11 mmol), N,N-dimethylacetamide (3.0 mL) and Cs₂CO₃ (112 mg, 0.34 mmol) were combined and warmed to 70° C. with stirring overnight. The mixture was concentrated, dissolved in 50 mL of 5% MeOH/DCM and 5 mL of saturated aqueous sodium bicarbonate solution and stirred for 5 minutes. The organic fraction was separated, concentrated and purified by column chromatography over silica gel using a gradient elution going from 8% MeOH to 15% MeOH in 1:1 EtOAc/hexane with 1% DMEA to provide 2-[4-(6,7-dimethoxyquinazolin-4-yl)-2-phenylpiperazin-1-yl]-N,N-dimethylethanamine as a colorless gum. ¹H NMR (CDCl₃) δ 8.68 (s, 1H), 7.57-7.54 (m, 2H), 7.36-7.22 (m, 4H) 6.89 (s, 1H), 5.39 (t, 1H), 4.35-4.09 (m, 5H), 3.99 (s, 3H), 3.70-3.65 (m, 2H), 3.52 (s, 3H), 3.34 (dt, 1H), 2.55 (t, 2H), 2.25 (s, 6H).

### Example 6

### Synthesis of 1′-(6,7-Dimethoxyquinazolin-4-yl)-1,3′-bipiperidin-2-one

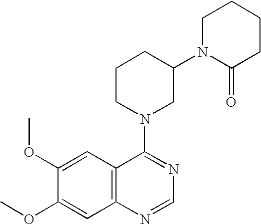

4-Chloro-6,7-dimethoxyquinazoline (83 mg, 0.37 mmol), 3-(N-delta-valerolactam)-piperidine hydrochloride (60.8 mg, 0.278 mmol), tetra-n-butylammonium iodide (31.6 mg, 0.086 mmol), potassium carbonate (116 mg, 0.84 mmol) and DMA (1.5 mL) were combined and sealed in a microwave reaction tube. The mixture was warmed in an oil bath to 140° C. for 2 h, cooled to room temperature and concentrated to a dark-brown oil. The residue was taken up water (15 mL) and the pH was adjusted to 9 by the addition of saturated aqueous NaHCO₃ and then extracted into EtOAc. The organic extracts were combined, dried (Na₂SO₄) and concentrated. The material was purified by chromatography over SiO₂ using a gradient elution going from 0% MeOH to 5% MeOH in DCM. Further purification using a Berger SFC mini-gram with attached 7.8 mm×250 mm pyridine column using 10% MeOH with 0.1% DME in CO₂ (1) as eluant with a flow rate of 9.9 mL/min provided 4.7 mg of 1′-(6,7-dimethoxyquinazolin-4-yl)-1,3′-bipiperidin-2-one as a white solid. ¹H NMR (CDCl₃) δ 8.68 (s, 1H), 7.25 (s, 1H), 7.24 (s, 1H), 4.99 (tt, 1H), 4.09 (s, 3H), 4.05 (m, 2H), 4.03 (s, 3H), 3.36-3.21 (m, 2H), 2.96-2.85 (m, 2H) 2.46-2.39 (m, 2H), 2.02-1.95 (m, 3H), 1.84-1.73 (m, 5H). LCMS: Retention time=2.54, M+H=371.2.

### Biological Examples

### Example 7

### mPDE10A7 Enzyme Activity and Inhibition

Enzyme Activity

To analyze the enzyme activity, 5 μL of serial diluted mPDE10A7 containing lysate were incubated with equal volumes of diluted (100-fold) fluorescein labeled cAMP or cGMP for 30 minutes in MDC HE 96-well assay plates (Molecular Devices Corp., Sunnyvale Calif.) at room temperature. Both the enzyme and the substrates were diluted in the following assay buffer: Tris/HCl (pH 8.0) 50 mM, MgCl₂ 5 mM, 2-mercaptoethanol 4 mM, BSA 0.33 mg/mL. After incubation, the reaction was stopped by adding 20 μL of diluted (400-fold) binding reagents and was incubated for an hour at room temperature. The plates were counted in an Analyst GT (Molecular Devices) for fluorescence polarization. An IMAP Assay kit (Molecular Devices was used to assess enzyme properties of mmPDE10A7. Data were analyzed with SOFTMAX PRO software (Molecular Devices).

Enzyme Inhibition

To check the inhibition profile, 10 μL of serial diluted compounds were incubated with 30 μl of diluted PDE enzymes in a 96-well polystyrene assay plate for 30 minutes at room temperature. After incubation, 5 μL of the compound-enzyme mixture were aliquoted into a MDC HE black plate, mixed with 5 μl of 100-fold diluted fluorescein labeled substrates (cAMP or cGMP), and incubated for 30 minutes at room temperature. The reaction was stopped by adding 20 μL of diluted binding reagents and counted in an Analyst GT for fluorescence polarization. The data were analyzed with SoftMax Pro. Compounds of the invention showed inhibited mPDE10A7 in this assay typically with IC₅₀ values of less than 5 μM.

### Example 8

### Apomorphine Induced Deficits in Prepulse Inhibition of the Startle Response in Rats an in vivo Test for Antipsychotic Activity

The thought disorders that are characteristic of schizophrenia may result from an inability to filter, or gate, sensorimotor information. The ability to gate sensorimotor information can be tested in many animals as well as in humans. A test that is commonly used is the reversal of apomorphine-induced deficits in the prepulse inhibition of the startle response. The startle response is a reflex to a sudden intense stimulus such as a burst of noise. In this example, rats are exposed to a sudden burst of noise, at a level of 120 db for 40 msec, e.g. the reflex activity of the rats is measured. The reflex of the rats to the burst of noise may be attenuated by preceding the startle stimulus with a stimulus of lower intensity, at 3 to 12 db above background (65 db), which will attenuate the startle reflex by 20 to 80%.

The prepulse inhibition of the startle reflex, described above, may be attenuated by drugs that affect receptor signaling pathways in the CNS. One commonly used drug is the dopamine receptor agonist apomorphine. Administration of apomorphine will reduce the inhibition of the startle reflex produced by the prepulse. Antipsychotic drugs such as haloperidol will prevent apomorphine from reducing the prepulse inhibition of the startle reflex. This assay may be used to test the antipsychotic efficacy of PDE10 inhibitors, as they reduce the apomorphine-induced deficit in the prepulse inhibition of startle.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

What is claimed:

1. A compound of Formula (I): 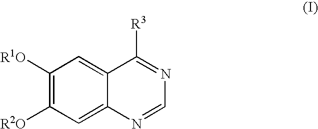
or an individual stereoisomer, a mixture of stereoisomers, or a pharmaceutically acceptable salt thereof, wherein:
R¹ and R² are each independently selected from hydrogen, alkyl, or haloalkyl; and
R³ is:
(i) phenyl, six-membered heteroaryl, or a monocyclic six- or seven-membered heterocyclyl ring substituted with:
R⁴, where R⁴ is hydrogen, alkyl, halo, haloalkyl, haloalkoxy, cycloalkyl, cycloalkylalkyl, aryl, heteroaryl, heterocyclyl, aralkyl, heteroaralkyl, heterocyclylalkyl, or —XR⁷ (where X is —O—, —CO—, —C(O)O—, —OC(O)—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and
R⁵ and R⁶, where R⁵ and R⁶ are each independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl provided that at least one of R⁴, R⁵ and R⁶ is not hydrogen;
and wherein the aromatic or alicyclic ring in R⁴, R⁵, R⁶, and R⁷ is optionally substituted with one to three substituents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substituents independently selected from R^{d} and R^{e} where R^{d} and R^{e} are hydrogen or fluoro;
(ii) pyrrolyl, pyrrolidinyl, 2,4-dioxoimidazolidinyl, or 2-oxopyrrolidinyl substituted with:
R¹³, where R¹³ is hydrogen, alkyl, halo, haloalkyl, haloalkoxy, cycloalkyl, cycloalkylalkyl, aryl, heteroaryl, heterocyclyl, aralkyl, heteroaralkyl, heterocyclylalkyl, or —XR¹⁶ (where X is —O—, —CO—, —OC(O)—, —C(O)O—, —NR¹⁷CO—, —CONR¹⁸—, —NR¹⁹—, —S—, —SO—, —SO₂—, —NR²⁰SO₂—, or —SO₂NR²¹— where R¹⁷-R²¹ are independently hydrogen, alkyl, hydroxyalkyl, alkoxyalkyl, aryl, aralkyl, heteroaryl, heteroaralkyl, acyl, or heterocyclylalkyl and R¹⁶ is cycloalkyl, cycloalkylalkyl, aryl, heteroaryl, heterocyclyl, aralkyl, heteroaralkyl, or heterocyclylalkyl); and
R¹⁴ and R¹⁵, where R¹⁴ and R¹⁵ are each independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino, aryl, heteroaryl or heterocyclyl provided that at least one of R¹³, R¹⁴ and R¹⁵ is not hydrogen; and
wherein the aromatic or alicyclic ring in R¹³, R¹⁴, R¹⁵, and R¹⁶ is optionally substituted with one to three substituents independently selected from R^{f}, R^{g}, and R^{h} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, or disubstituted amino, optionally substituted phenyl, optionally substituted heteroaryl, or optionally substituted heterocyclyl; and additionally substituted with one or two substituents independently selected from Rⁱ and R^{j} where Rⁱ and R^{j} are hydrogen or fluoro;
(iii) a ring of formula (a) 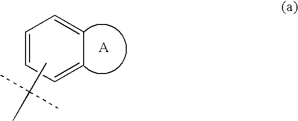
where A is a monocyclic saturated five-, six-, or seven membered heterocyclyl ring and the ring (a) is substituted with:
R²², where R²² is selected from hydrogen, alkyl, haloalkyl, haloalkoxy, cycloalkyl, cycloalkylalkyl, aryl, heteroaryl, heterocyclyl, aralkyl, heteroaralkyl, heterocyclylalkyl, or —XR²⁵ (where X is —O—, —CO—, —C(O)O—, —OC(O)—, —NR²⁶CO—, —CONR²⁷—, —NR²⁸—, —S—, —SO—, —SO₂—, —NR²⁹SO₂—, or —SO₂NR³⁰— where R²⁶-R³⁰ are independently hydrogen, alkyl, hydroxyalkyl, alkoxyalkyl, aryl, aralkyl, heteroaryl, heteroaralkyl, acyl, or heterocyclylalkyl and R²⁵ is hydrogen, alkyl, haloalkyl, cycloalkyl, cycloalkylalkyl, aryl, heteroaryl, heterocyclyl, aralkyl, heteroaralkyl, or heterocyclylalkyl); and
R²³ and R²⁴, where R²³ and R²⁴ are each independently selected from hydrogen, alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino, aryl, heteroaryl or heterocyclyl; and
wherein the aromatic or alicyclic ring in R²², R²³, R²⁴, and R²⁵ is optionally substituted with one to three substituents independently selected from R^{k}, R^{l}, and R^{m} which alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, or disubstituted amino, optionally substituted phenyl, optionally substituted heteroaryl, or optionally substituted heterocyclyl; and additionally substituted with one or two substituents independently selected from Rⁿ and R^{o} where Rⁿ and R^{o} are hydrogen or fluoro provided that at least one of R²², R²³ and R²⁴ is not hydrogen; or
(iv) a ring of formula (b), (c), or (d): 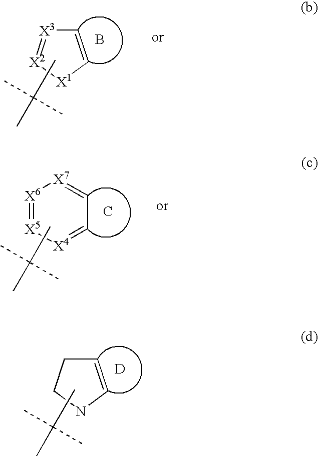
where:
X¹, X², and X³ are each independently carbon, nitrogen, oxygen or sulfur, provided that at least two of X¹, X², and X³ are other than carbon;
X⁴, X⁵, X⁶ and X⁷ are each independently carbon or nitrogen, provided that at least two of X⁴, X⁵, X⁶ and X⁷ are other than carbon; and
B, C, and D are phenyl, a five- or six-membered heteroaryl ring (wherein the five-membered heteroaryl ring contains one or two heteroatoms independently selected from nitrogen, oxygen, and sulfur and the six-membered heteroaryl ring contains one or two nitrogen atoms, the rest of the ring atoms being carbon), or a five-, six-, or seven-membered heterocyclyl ring; and wherein rings (b) and (c) are substituted with:
R³¹, where R³¹ is cycloalkyl, cycloalkylalkyl, aryl, heteroaryl, heterocyclyl, aralkyl, heteroaralkyl, heterocyclylalkyl, or —XR³⁴ (where X is —O—, —CO—, —OC(O)—, —C(O)O—, —NR³⁵CO—, —CONR³⁶—, —NR³⁷—, —S—, —SO—, —SO₂—, —NR³⁸SO₂—, or —SO₂NR³⁹— where R³⁵-R³⁹ are independently hydrogen, alkyl, hydroxyalkyl, alkoxyalkyl, aryl, aralkyl, heteroaryl, heteroaralkyl, acyl, or heterocyclylalkyl and R³⁴ is cycloalkyl, cycloalkylalkyl, aryl, heteroaryl, heterocyclyl, aralkyl, heteroaralkyl, or heterocyclylalkyl); and
R³² and R³³, where R³² and R³³ are each independently selected from hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino, aryl, heteroaryl or heterocyclyl provided that at least one of R³¹, R³² and R³³ is not hydrogen; and
wherein the aromatic or alicyclic ring in R³¹, R³², R³³, and R³⁴ is optionally substituted with one to three substituents independently selected from R^{p}, R^{q}, and R^{r} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfonyl, monosubstituted amino, or, disubstituted amino, optionally substituted phenyl, optionally substituted heteroaryl, or optionally substituted heterocyclyl; and additionally substituted with one or two substituents independently selected from R^{s} and R^{t} where R^{s} and R^{t} are hydrogen or fluoro; and
provided that:
(i) R³ is not disubstituted piperidin-1-yl where one substituent is substituted or unsubstituted aryl or heteroaryl, and the other substituent is alkyl, carboxy, alkoxycarbonyl, cyano, hydroxyl, alkoxy, haloalkoxy, pyridin-2-yloxy, —COR, —CONRR′, —COOR, —OR or —NRR′ (where R and R′ are independently hydrogen, alkyl, or unsubstituted aryl), or —NHCOR (where R is alkyl, haloalkyl, or unsubstituted aryl);
(ii) when R³ is pyrrolidin-1-yl, R¹³ is not —XR¹⁶ where X is —O— and R¹⁶ is substituted or unsubstituted aryl or heteroaryl;
(iii) R³ is not (a) monosubstituted phenyl wherein the substituent is hydroxy, nitro, halo, alkoxy, haloalkyl, or unsubstituted aryl; (b) disubstituted phenyl wherein the substituents are independently selected from halo or alkoxy; (c) monosubstituted pyridinyl wherein the substituent is selected from halo or alkoxy; (d) 2,6-dimethylmorpholinyl, or (e) 5-amino-2-phenylcarbonylaminopyrimidinyl;
(iv) R³ is not monosubstituted piperidinyl wherein the substituent is alkyl, hydroxy, carboxy, alkoxycarbonyl, hydroxyalkyl, haloalkyl, substituted or unsubstituted aryl or heteroaryl, substituted or unsubstituted, saturated or unsaturated heterocyclyl or heterocyclylalkyl wherein the heterocyclyl ring contains two ring atoms that are heteroatom selected from N, O, or S(O)ₙ, where n is an integer from 0 to 2, the remaining ring atoms being C, where one or two ring carbon atoms can optionally be replaced by a —CO— group and the heterocyclic ring is optionally fused to a phenyl provided that when the heterocyclyl ring is bicyclic, the bicyclic heterocyclyl ring is attached to the piperidinyl ring via the non-phenyl portion of the ring; —COR (where R is alkyl or unsubstituted aryl), —COOR (where R is unsubstituted aryl), —CONRR′ (where R is hydrogen, alkyl, or unsubstituted aryl, and R is unsubstituted aryl), —NRCOR′ (where R is hydrogen, alkyl, or unsubstitued aryl and R′ is alkyl, haloalkyl, unsubstituted aryl, 4-acetylaminophenyl, piperidin-1-yl, piperidin-1-ylalkyl, or pyridinyl); —NRSO₂R′ (where R is hydrogen or alkyl and R′ is alkyl, 4-acetylaminophenyl, or pyridinyl); —NRR′ (where R is hydrogen, alkyl, or unsubstituted aryl and R′ is alkyl, 2-aminoethyl, 2-benzylaminoethyl, unsubstituted aryl, or pyridinylmethyl); -(alkylene)NRR′ ([where R is hydrogen or alkyl and R′ is hydrogen, alkyl or —COR″ where R″ is alkyl); phenyl (optionally substituted with haloalkyl or alkoxy); substituted or unsubstituted indolinyl, oxazolyl, benzo[d]oxazolyl, oxiranyl, 1H-benzo[d]imidazolyl, 1H-benzo[d][1,2,3]triazolyl, pyridin-2-yloxy, tetrahydronaphthalenyl, or 4H-1,2,4-triazolylalkyl; or piperidin-4-ylalkyl substituted with dialkoxyquinazoline;
(v) R³ is not disubstituted piperidinyl where one of the substituents is alkyl or hydroxy and the other substitutent is hydroxyalkyl, haloalkyl, 1,1-dioxoisothiazolidinylalkyl or 1H-benzo[d]imidazolyl-2(3H)-one, wherein each of these rings is optionally substituted with one or two alkyl, and the 1H-benzo[d]imidazolyl-2(3H)-one is attached to the piperidinyl ring via the non-phenyl portion of the ring;
(vi) R³ is not monosubstituted or disubstituted piperazin-4-yl or homopiperazin-4-yl where the substitutent(s) is alkyl; or R³ is not piperazin-4-yl or homopiperazin-4-yl where R⁵ is hydrogen, R⁶ is hydrogen or alkyl and R⁴ is other than hydrogen and at least one of R⁴ and R⁶ is located at the N−1 nitrogen of the piperazine or homopiperazine ring.

2. The compound of claim 1 wherein R¹ and R² are alkyl.

3. The compound of claim 1 wherein one of R¹ and R² is alkyl and the other is haloalkyl.

4. The compound of claim 1 wherein R³ is a monocyclic six- or seven-membered heterocyclyl ring.

5. The compound of claim 1 wherein R³ is a ring of formula: 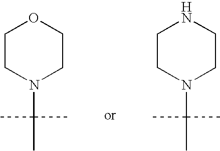
each substituted with R⁴, R⁵ and R⁶.

6. The compound of claim 5 where R³ is a ring of formula: 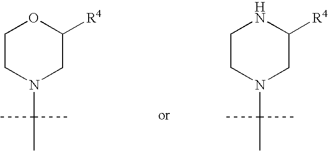
where R⁴ is phenyl substituted with R^{a}, R^{b} and R^{c} provided that one of R^{a}, R^{b} and R^{c} is not hydrogen.

7. The compound of claim 5 where R³ is a ring of formula: 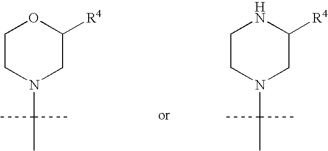
wherein R⁴ is —NHCOR⁷ where R⁷ is phenyl optionally substituted with R^{a}, R^{b} and R^{c} as defined in the Summary of the Invention provided that one of R^{a}, R^{b} and R^{c} is not hydrogen.

8. The compound of any of the claim 5 wherein R³ is a ring of formula: 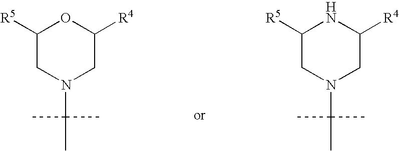
where:
R⁴ is phenyl optionally substituted with R^{b} and R^{c} wherein R^{a}, R^{b}, and R^{c} provided that one of R^{a}, R^{b} and R^{c} is not hydrogen.

9. The compound of claim 2 wherein R³ is a ring of formula: 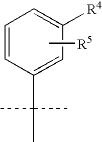
where one of R⁴ and R⁵ is hydrogen, alkyl, halo, haloalkyl, alkoxy, haloalkoxy, cyano, amino, monsubstituted or disubstituted amino, or —XR⁷ (where X is —O—, —CO—, —OC(O)—, —C(O)O, —NR⁸CO—, —CONR⁹—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); and the other of R⁴ and R⁵ is aryl, heteroaryl, or heterocyclyl; and wherein the aromatic or alicyclic ring in R⁴ and R⁵ is optionally substituted with one to three substitutents independently selected from R^{a}, R^{b}, and R^{c}.

10. The compound of claims 2 wherein R³ is a ring of formula: 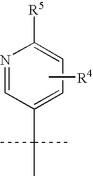
where one of R⁴ and R⁵ is hydrogen, alkyl, halo, haloalkyl, alkoxy, haloalkoxy, cyano, amino, monsubstituted or disubstituted amino, or —XR⁷ (where X is —O—, —CO—, —OC(O)—, —C(O)O, —NR⁸CO—, —CONR⁹—, —S—, —SO—, —SO₂—, —NR¹SO₂—, or —SO₂NR¹²— where R⁸-R² are independently hydrogen, alkyl, hydroxyalkyl, alkoxyalkyl, aryl, aralkyl, heteroaryl, heteroaralkyl, acyl, or heterocyclylalkyl and R⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); and the other of R⁴ and R⁵ is aryl, heteroaryl, or heterocyclyl; and wherein the aromatic or alicyclic ring in R⁴ and R⁵ is optionally substituted with one to three substitutents independently selected from R^{a}, R^{b} and R^{c}.

11. The compound of claim 2 wherein R³ is a ring of formula: 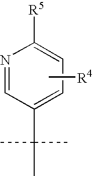
where R⁴ is hydrogen, alkyl, or fluoro and R⁵ is heterocyclyl, monosubstituted or disubstituted amino, wherein the aromatic or alicyclic ring in R⁵ is optionally substituted with one to three substitutents independently selected from R^{a}, R^{b}, and R^{c}.

12. The compound of claim 1 where R³ is:
(i) phenyl or six-membered heteroaryl substituted with:
R⁴, where R⁴ is hydrogen, alkyl, halo, haloalkyl, haloalkoxy, cycloalkyl, cycloalkylalkyl, aryl, heteroaryl, heterocyclyl, aralkyl, heteroaralkyl, heterocyclylalkyl, or —XR⁷ (where X is —O—, —CO—, —C(O)O—, —OC(O)—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and
R⁵ and R⁶, where R⁵ and R⁶ are each independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl provided that at least one of R⁴, R⁵ and R⁶ is not hydrogen;
and wherein the aromatic or alicyclic ring in R⁴, R⁵, R⁶, and R⁷ is optionally substituted with one to three substituents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substituents independently selected from R^{d} and R^{e} where R^{d} and R^{e} are hydrogen or fluoro;
(iii) a ring of formula (a) 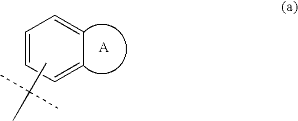
where A is a monocyclic saturated five-, six-, or seven membered heterocyclyl ring and the ring (a) is substituted with:
R²², where R²² is selected from hydrogen, alkyl, haloalkyl, haloalkoxy, cycloalkyl, cycloalkylalkyl, aryl, heteroaryl, heterocyclyl, aralkyl, heteroaralkyl, heterocyclylalkyl, or —XR²⁵ (where X is —O—, —CO—, —C(O)O—, —OC(O)—, —NR²⁶CO—, —CONR²⁷—, —NR²¹, —S—, —SO—, —SO₂—, —NR²⁹SO₂—, or —SO₂NR³⁰— where R²⁶-R³⁰ are independently hydrogen, alkyl, hydroxyalkyl, alkoxyalkyl, aryl, aralkyl, heteroaryl, heteroaralkyl, acyl, or heterocyclylalkyl and R²⁵ is hydrogen, alkyl, haloalkyl, cycloalkyl, cycloalkylalkyl, aryl, heteroaryl, heterocyclyl, aralkyl, heteroaralkyl, or heterocyclylalkyl); and
R²³ and R²⁴, where R²³ and R²⁴ are each independently selected from hydrogen, alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino, aryl, heteroaryl or heterocyclyl; and
wherein the aromatic or alicyclic ring in R²², R²³, R²⁴, and R²⁵ is optionally substituted with one to three substituents independently selected from R^{k}, R^{l}, and R^{m} which alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino, optionally substituted phenyl, optionally substituted heteroaryl, or optionally substituted heterocyclyl; and additionally substituted with one or two substituents independently selected from Rⁿ and R^{o} where Rⁿ and R^{o} are hydrogen or fluoro provided that at least one of R²², R²³ and R²⁴ is not hydrogen; or
(iv) a ring of formula (b) or (c): 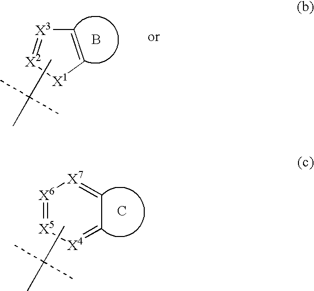
where:
X¹, X², and X³ are each independently carbon, nitrogen, oxygen or sulfur, provided that at least two of X¹, X², and X³ are other than carbon;
X⁴, X⁵, X⁶ and X⁷ are each independently carbon or nitrogen provided that at least two of X⁴, X⁵, X⁶ and X⁷ are other than carbon; and
B and C are phenyl, a five- or six-membered heteroaryl ring (wherein the five-membered heteroaryl ring contains one or two heteroatoms independently selected from nitrogen, oxygen, and sulfur and the six-membered heteroaryl ring contains one or two nitrogen atoms, the rest of the ring atoms being carbon), or a five-, six-, or seven-membered heterocyclyl ring; and wherein rings (b) and (c) are substituted with:
R³¹, where R³¹ is cycloalkyl, cycloalkylalkyl, aryl, heteroaryl, heterocyclyl, aralkyl, heteroaralkyl, heterocyclylalkyl, or —XR³⁴ (where X is —O—, —CO—, —OC(O)—, —C(O)O—, —NR³⁵CO—, —CONR³⁶—, —NR³⁷—, —S—, —SO—, —SO₂—, —NR³⁸SO₂—, or —SO₂NR³⁹— where R³⁵-R³⁹ are independently hydrogen, alkyl, hydroxyalkyl, alkoxyalkyl, aryl, aralkyl, heteroaryl, heteroaralkyl, acyl, or heterocyclylalkyl and R³⁴ is cycloalkyl, cycloalkylalkyl, aryl, heteroaryl, heterocyclyl, aralkyl, heteroaralkyl, or heterocyclylalkyl); and
R³² and R³³, where R³² and R³³ are each independently selected from hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino, aryl, heteroaryl or heterocyclyl provided that at least one of R³¹, R³² and R³³ is not hydrogen; and
wherein the aromatic or alicyclic ring in R³¹, R³², R³³, and R³⁴ is optionally substituted with one to three substituents independently selected from R^{p}, R^{q}, and R^{r} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfonyl, monosubstituted amino, or, disubstituted amino, optionally substituted phenyl, optionally substituted heteroaryl, or optionally substituted heterocyclyl; and additionally substituted with one or two substituents independently selected from R^{s} and R^{t} where R^{s} and R^{t} are hydrogen or fluoro.

13. A pharmaceutical composition comprising at least a compound of claim 1 and a pharmaceutically acceptable expicient.

14. A method of treating a disorder treatable by inhibition of PDE10 in a patient which method comprises administering to the patient a pharmaceutical composition comprising a compound of Formula (I): 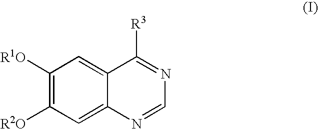
or an individual stereoisomer, a mixture of stereoisomers, or a pharmaceutically acceptable salt thereof; or mixtures thereof, wherein:
R¹ and R² are each independently selected from hydrogen, alkyl, or haloalkyl; and
R³ is:
(i) phenyl, six-membered heteroaryl, or a monocyclic six- or seven-membered heterocyclyl ring substituted with:
R⁴, where R⁴ is hydrogen, alkyl, halo, haloalkyl, haloalkoxy, cycloalkyl, cycloalkylalkyl, aryl, heteroaryl, heterocyclyl, aralkyl, heteroaralkyl, heterocyclylalkyl, or —XR⁷ (where X is —O—, —CO—, —C(O)O—, —OC(O)—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and
R⁵ and R⁶, where R⁵ and R⁶ are each independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl provided that at least one of R⁴, R⁵ and R⁶ is not hydrogen;
and wherein the aromatic or alicyclic ring in R⁴, R⁵, R⁶, and R⁷ is optionally substituted with one to three substituents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substituents independently selected from R^{d} and R^{e} where R^{d} and R^{e} are hydrogen or fluoro;
(ii) pyrrolyl, pyrrolidinyl, 2,4-dioxoimidazolidinyl, or 2-oxopyrrolidinyl substituted with:
R¹³, where R¹³ is hydrogen, alkyl, halo, haloalkyl, haloalkoxy, cycloalkyl, cycloalkylalkyl, aryl, heteroaryl, heterocyclyl, aralkyl, heteroaralkyl, heterocyclylalkyl, or —XR¹⁶ (where X is —O—, —CO—, —OC(O)—, —C(O)O—, —NR¹⁷CO—, —CONR¹⁸—, —NR¹⁹—, —S—, —SO—, —SO₂—, —NR²¹SO₂—, or —SO₂NR²¹— where R¹⁷-R²¹ are independently hydrogen, alkyl, hydroxyalkyl, alkoxyalkyl, aryl, aralkyl, heteroaryl, heteroaralkyl, acyl, or heterocyclylalkyl and R¹⁶ is cycloalkyl, cycloalkylalkyl, aryl, heteroaryl, heterocyclyl, aralkyl, heteroaralkyl, or heterocyclylalkyl); and
R¹⁴ and R¹⁵, where R¹⁴ and R¹⁵ are each independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino, aryl, heteroaryl or heterocyclyl provided that at least one of R¹³, R¹⁴ and R¹⁵ is not hydrogen; and
wherein the aromatic or alicyclic ring in R³, R⁴, R¹⁵, and R¹⁶ is optionally substituted with one to three substituents independently selected from R^{f}, R^{g}, and R^{h} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino, optionally substituted phenyl, optionally substituted heteroaryl, or optionally substituted heterocyclyl; and additionally substituted with one or two substituents independently selected from Rⁱ and R^{j} where Rⁱ and R^{j} are hydrogen or fluoro;
(iii) a ring of formula (a) 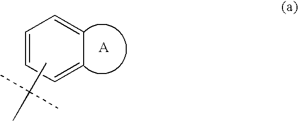
where A is a monocyclic saturated five-, six-, or seven membered heterocyclyl ring and the ring (a) is substituted with:
R²², where R²² is selected from hydrogen, alkyl, haloalkyl, haloalkoxy, cycloalkyl, cycloalkylalkyl, aryl, heteroaryl, heterocyclyl, aralkyl, heteroaralkyl, heterocyclylalkyl, or —XR²⁵ (where X is —O—, —CO—, —C(O)O—, —OC(O)—, —NR²⁶CO—, —CONR²⁷—, —NR²⁸—, —S—, —SO—, —SO₂—, —NR²⁹SO₂—, or —SO₂NR³¹— where R²⁶-R³⁰ are independently hydrogen, alkyl, hydroxyalkyl, alkoxyalkyl, aryl, aralkyl, heteroaryl, heteroaralkyl, acyl, or heterocyclylalkyl and R²⁵ is hydrogen, alkyl, haloalkyl, cycloalkyl, cycloalkylalkyl, aryl, heteroaryl, heterocyclyl, aralkyl, heteroaralkyl, or heterocyclylalkyl); and
R²³ and R²⁴, where R²³ and R²⁴ are each independently selected from hydrogen, alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino, aryl, heteroaryl or heterocyclyl; and
wherein the aromatic or alicyclic ring in R²², R²³, R²⁴, and R²⁵ is optionally substituted with one to three substituents independently selected from R^{k}, R^{l}, and R^{m} which alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino, optionally substituted phenyl, optionally substituted heteroaryl, or optionally substituted heterocyclyl; and additionally substituted with one or two substituents independently selected from Rⁿ and R^{o} where Rⁿ and R^{o} are hydrogen or fluoro provided that at least one of R²², R²³ and R²⁴ is not hydrogen; or
(iv) a ring of formula (b), (c), or (d): 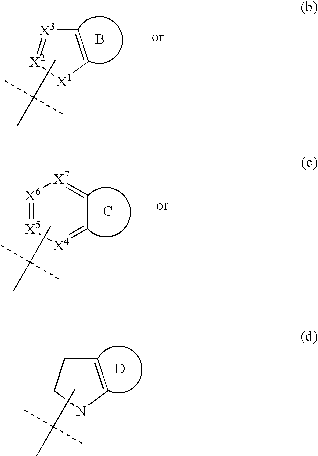
where:
X¹, X², and X³ are each independently carbon, nitrogen, oxygen or sulfur, provided that at least two of X¹, X², and X³ are other than carbon;
X⁴, X⁵, X⁶ and X⁷ are each independently carbon or nitrogen provided that at least two of X⁴, X⁵, X⁶ and X⁷ are other than carbon; and
B, C, and D are phenyl, a five- or six-membered heteroaryl ring (wherein the five-membered heteroaryl ring contains one or two heteroatoms independently selected from nitrogen, oxygen, and sulfur and the six-membered heteroaryl ring contains one or two nitrogen atoms, the rest of the ring atoms being carbon), or a five-, six-, or seven-membered heterocyclyl ring; and wherein rings (b) and (c) are substituted with:
R³¹, where R³¹ is cycloalkyl, cycloalkylalkyl, aryl, heteroaryl, heterocyclyl, aralkyl, heteroaralkyl, heterocyclylalkyl, or —XR³⁴ (where X is —O—, —CO—, —OC(O)—, —C(O)O—, —NR³⁵CO—, —CONR³⁶—, —NR³⁷—, —S—, —SO—, —SO₂—, —NR³⁸SO₂—, or —SO₂NR³⁹— where R³⁵-R³⁹ are independently hydrogen, alkyl, hydroxyalkyl, alkoxyalkyl, aryl, aralkyl, heteroaryl, heteroaralkyl, acyl, or heterocyclylalkyl and R³⁴ is cycloalkyl, cycloalkylalkyl, aryl, heteroaryl, heterocyclyl, aralkyl, heteroaralkyl, or heterocyclylalkyl); and
R³² and R³³, where R³² and R³³ are each independently selected from hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino, aryl, heteroaryl or heterocyclyl provided that at least one of R³¹, R³² and R³³ is not hydrogen; and
wherein the aromatic or alicyclic ring in R³¹, R³², R³³, and R³⁴ is optionally substituted with one to three substituents independently selected from R^{p}, R^{q}, and R^{r} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfonyl, monosubstituted amino, or, disubstituted amino, optionally substituted phenyl, optionally substituted heteroaryl, or optionally substituted heterocyclyl; and additionally substituted with one or two substituents independently selected from R^{s} and R^{t} where R^{s} and R^{t} are hydrogen or fluoro; and
provided that:
(i) R³ is not disubstituted piperidin-1-yl where one substituent is substituted or unsubstituted aryl or heteroaryl, and the other substituent is alkyl, carboxy, alkoxycarbonyl, cyano, hydroxyl, alkoxy, haloalkoxy, pyridin-2-yloxy, —COR, —CONRR′, —COOR, —OR or —NRR′ (where R and R′ are independently hydrogen, alkyl, or unsubstituted aryl), or —NHCOR (where R is alkyl, haloalkyl, or unsubstituted aryl); or
(ii) when R³ is pyrrolidin-1-yl, R¹³ is not —XR¹⁶ where X is —O— and R¹⁶ is substituted or unsubstituted aryl or heteroaryl.

15. The method of claim 14 where the disease is schizophrenia, bipolar disorder, and obsessive-compulsive disorder.
